(19) United States
(12) Patent Application Publication
(10) Pub. No.: US 2008/0275053 A1
(43) Pub. Date: Nov. 6, 2008

(54) Heterocyclyl Compounds

(75) Inventors: Gerard Martin Paul Giblin, Essex (GB); Adrian Hall, Essex (GB); Ian Reginald Kilford, Essex (GB); Xiao Qing Lewell, Hertfordshire (GB); Neil Derek Miller, Essex (GB); Alan Naylor, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GlaxoSmithKline

(21) Appl. No.: 10/575,072

(22) PCT Filed: Oct. 6, 2004
(86) PCT No.: PCT/EP04/11366

§ 371 Date: May 22, 2006

(30) Foreign Application Priority Data
Oct. 8, 2003 (GB) 0323585.0

(51) Int. Cl. A61K 31/4965; A61P 25/00; A61P 29/00; A61P 37/00; C07D 207/337; C07D 231/12; C07D 241/12; A61K 31/4418; A61K 31/4439; A61K 31/365; C07D 307/58; C07D 213/55; C07D 471/04; A61K 31/40; A61K 31/415

(52) U.S. Cl. 514/252.1; 514/406; 514/427; 548/563; 548/377.1; 544/336; 549/323; 546/342; 546/276.4; 514/473; 514/277; 514/343

(57) ABSTRACT

Compounds of formula (I) or a pharmaceutically acceptable derivative thereof:

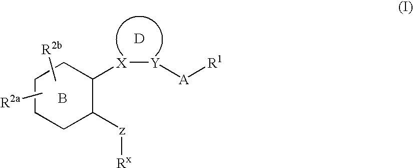

wherein A, B, D, Z, R¹, R^{2a}, R^{2b}, and R^{x} are as defined in the specification, a process for the preparation of such compounds, pharmaceutical compositions comprising such compounds and the use of such compounds in medicine.

This invention relates to heterocyclyl compounds, to processes for their preparation, to pharmaceutical compositions containing them and to their use in medicine, in particular their use in the treatment of conditions mediated by the action of PGE₂ at EP₁ receptors.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Madouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have ant-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421-A1 (Jan. 8, 1997), WO 01/19814 (22 Mar. 2001), WO 03/084917 (16 Oct. 2003), WO 03/101959 (11 Dec. 2003) and WO 2004/039753 (13 May 2004) disclose compounds as being useful in the treatment of prostaglandin mediated diseases.

It is now suggested that a novel group of heterocyclyl derivatives surprisingly are selective for the EP₁ receptor over the EP₃ receptor, and are therefore indicated to be useful in treating conditions mediated by the action of PGE₂ at EP₁ receptors. Such conditions include pain, or inflammatory, immunological, bone, neurodegenerative or renal disorders.

Accordingly the present invention provides compounds of formula (I):

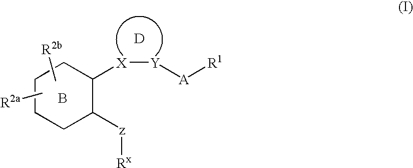

wherein:
A represents an optionally substituted aryl, or an optionally substituted 5 or 6-membered heterocyclyl ring, or an optionally substituted bicyclic heterocyclyl group;
B represents a phenyl or pyridyl ring;
D represents an optionally substituted 5- or 6-membered heterocycyl ring containing one or two heteroatoms selected from N, S and O, wherein X and Y are each independently selected from N and C;
Z represents O, S, SO, or SO₂;
R¹ represents CO₂H, CN, CONR⁵R⁶, CH₂CO₂H, optionally substituted alkyl, optionally substituted alkenyl, optionally substituted SO₂alkyl, SO₂NR⁶R⁸, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted CQ^{a}Q^{b}-heterocycle, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
Q^{a} and Q^{b} are each independently selected from hydrogen and CH₃;
wherein when A is a 6-membered ring the R¹ substituent and the D ring are attached to carbon atoms 1,2-, 1,3 or 1,4-relative to each other, and when A is a five-membered ring or bicyclic heterocyclyl group the R¹ substituent and the D ring are attached to substitutable carbon atoms 1,2- or 1,3 relative to each other;
and derivatives thereof;
provided that D is not imidazolyl, thienyl,

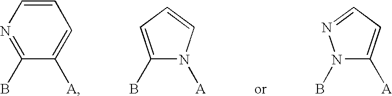

wherein A and B are as hereinbefore defined.

In one aspect X and Y are each C.

Suitably A includes pyridyl or optionally substituted phenyl.

An example of A includes optionally substituted phenyl.

Optional substituents for A include NHCOC₁₋₄alkyl.

When B is pyridyl, preferably the pyridine N atom is situated adjacent to the ring carbon carrying either the cyclopentene or Z substituent.

Suitably D includes:

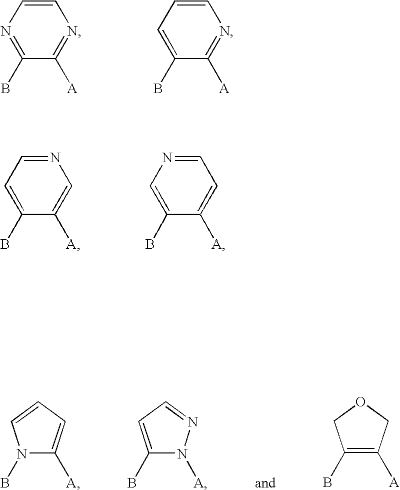

all of which may be optionally substituted.

Optional substituents for D include C₁₋₄alkyl and oxo. D may be substituted by up to four optional substituents, for example one or two substituents.

Preferably D includes:

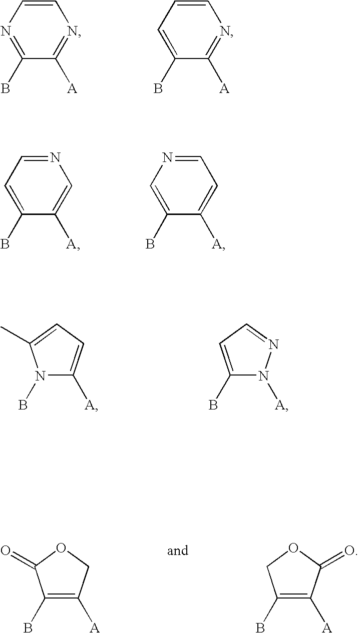

Suitably R¹ includes CO₂H.

Preferably Z is O,

Suitably R^{2a} and R^{2b} are selected from hydrogen, halo, and optionally substituted C₁₋₆alkyl, e.g. CF₃.

Preferably R^{2a} is hydrogen.

Preferably R^{2b} represents hydrogen, halo, or CF₃.

Preferably R^{2b} is positioned 1,4-relative to the Z substituent and 1,3-relative to the D ring.

Suitably R⁴ is hydrogen or C₁₋₄alkyl.

Suitably R⁵ includes hydrogen or C₁₋₄alkyl.

Suitably R⁶ includes hydrogen, and C₁₋₄alkyl.

Suitably R⁷ includes hydrogen or C₁₋₄alkyl.

Suitably R^{x} when an optionally substituted alkyl group includes C₁₋₈alkyl.

Suitably R^{x} is CH₂phenyl optionally substituted by one, two or three substituents selected from halogen.

A suitable example of Qₐ is hydrogen.

A suitable example of Q_{b} is hydrogen.

Compounds of formula (I) include:
3-{1-[2-(Benzyloxy)-phenyl]-5-methyl-1H-pyrrol-2-yl}-benzoic acid;
3-{1-[2-(Benzyloxy)-5-chloro-phenyl]-5-methyl-1H-pyrrol-2-yl}-benzoic acid;
3-{1-[2-(Benzyloxy)-5-bromo-phenyl]-5-methyl-1H-pyrrol-2-yl}-benzoic acid;
3-{5-[2-(Benzyloxy)phenyl]-1H-pyrazol-1-yl}-benzoic acid;
3-{5-[2-(Benzyloxy)-5-chloro-phenyl]-1H-pyrazol-1-yl}-benzoic acid;
3-{3-[2-(Benzyloxy)-chloro-phenyl]-pyridin-2-yl}-benzoic acid;
3-{4-[2-(Benzyloxy)-5-chloro-phenyl]-2-oxo-2,5-dihydro-furan-3-yl}-benzoic acid;
3-{3-[2-(Benzyloxy)₅-chloro-phenyl]-2-oxo-2,5-dihydrofuran-4-yl}-benzoic acid;
3-{3-[2-(Benzyloxy)-chloro-phenyl]-pyridin-4-yl}-benzoic acid;
3-{3-[2-(Benzyloxy)-phenyl]-pyridin-4-yl}-benzoic acid;
3-{4-[2-(Benzyloxy)-chloro-phenyl]-pyridin-3-yl}-benzoic acid;
3-{3-[2-(Benzyloxy)-chloro-phenyl]-pyridin-2-yl}-benzoic acid;
3-{3-[2-(Benzyloxy)-5-(trifluoromethyl-phenyl]-4-yl}-5-(acetylamino)-benzoic aid;
3-{3-[2-(4-Fluoro-benzyloxy)-5-trifluoromethyl)-phenyl]-pyridin)-4-yl}-acetylamino) benzoic acid;
3-{3-[2-(2,4-Difluoro benzyloxy)-5-(trifluoromethyl)-phenyl]-pyridin-4-yl}-5-(acetylamino) benzoic acid; and
3-{3-[2-(Benzyloxy)-phenyl]-pyridin-4-yl}-5-(acetylamino)-benzoic acid;
   and derivatives thereof.

A further compound of formula (I) is 6-{1-[2-(Benzyloxy)-5-chloro-phenyl]-5-methyl-1H-pyrrol-2-yl}2-pyridinecarboxylic acid and derivatives thereof.

The compounds of the invention are selective for EP₁ over EP₃.

Derivatives of the compounds of formula (I) include pharmaceutically acceptable derivatives.

The invention is described using the following definitions unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. A particular salt is the sodium salt. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tromethamine, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen or halo” are used to represent fluorine, chlorine, bromine or iodine, more preferably fluorine, chlorine and bromine.

The terms “alkyl” as a group or part of a group means a straight, branched or cyclic chain alkyl group or combinations thereof. Unless hereinbefore defined, examples of alkyl include C₁alkyl, for example methyl, ethyl, n-propyl, iso-propyl, n-butyl, sec-butyl, iso-butyl, tert-butyl, pentyl, hexyl, 1,1-dimethylethyl, cyclopentyl or cyclohexyl or combinations thereof.

The term “alkoxy” as a group or as part of a group means a straight, branched or cyclic chain alkoxy group. Unless hereinbefore defined examples of alkoxy include C₁₋₈alkoxy, for example methoxy, ethoxy, n-propoxy, iso-propoxy, n-butoxy, sec-butoxy, iso-butoxy, tert-butoxy, pentoxy, hexyloxy, cyclopropoxy, cyclobutoxy, cyclopentoxy or cyclohexyloxy.

The term “alkenyl” means linear or branched structures and combinations thereof, of the indicated number of carbon atoms, having at least one carbon-to-carbon double bond, wherein hydrogen may be replaced by an additional carbon to carbon double bond. C₂₋₈alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₂₋₈alkynyl, for example, includes ethynyl, propynyl, butynyl and the like.

The term “heterocyclyl” as a group or as part of a group unless hereinbefore defined means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocycyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocycyl groups are pyridyl, pyridazinyl, pyrimidinyl, pyrazinyl, triazinyl or tetrazinyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

The term “aryl” as a group or as part of a group means a 5- or 6 membered aromatic ring for example phenyl, or a 7 to 12 membered bicyclic ring system where at least one of the rings is aromatic, for example naphthyl. An aryl group may be substituted by up to four, preferably one to three substituents. Preferably the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents for example one or two substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

Optional substituents for alkyl, alkenyl or alkynyl groups unless hereinbefore defined include OH, CO₂H, CO₂C₁₋₆alkyl, NH₂, alkyl, NH₂, (O), OC, alkyl, phenyl or halo e.g. Cl, Br or F. An alkyl, alkenyl or alkynyl group may be substituted by one or more optional substituents, for example up to 5, 4, 3, 2 or 1 optional substituents. Particular substituted alkyl groups include those substituted by one or more fluorine atoms, up to per-fluorination, e.g. CH₂F, CHF₂, CF₃, C₂F₅, CH₂CF₃, and CH₂CH₂CF₃.

Optional substituents for alkoxy groups unless hereinbefore defined include OH, and halo e.g. Cl, Br or F. An alkoxy group may be substituted by one or more optional substituents, for example up to 5, 4, 3, or 2 optional substituents.

Optional substituents for aryl, heteroaryl or heterocyclyl groups, unless hereinbefore defined, include one or two substituents selected from halogen; C₁alkyl; and Cl alkoxy.

When the heteroatom nitrogen replaces a carbon atom in a C₁alkyl group, or when nitrogen is present in a heteroaryl, heterocyclyl or bicyclic heterocyclyl group the nitrogen atom will, where appropriate be substituted by one or two substituents selected from hydrogen and C₁₋alkyl, preferably hydrogen and C₁₋₆alkyl, more preferably hydrogen.

Compounds of formula (I) can be prepared as set forth in the following schemes and in the examples. The following processes form another aspect of the present invention.

For example, compounds of formula (Ia) which are compounds of formula (I) wherein X and Y are each C may be prepared by the general route below.

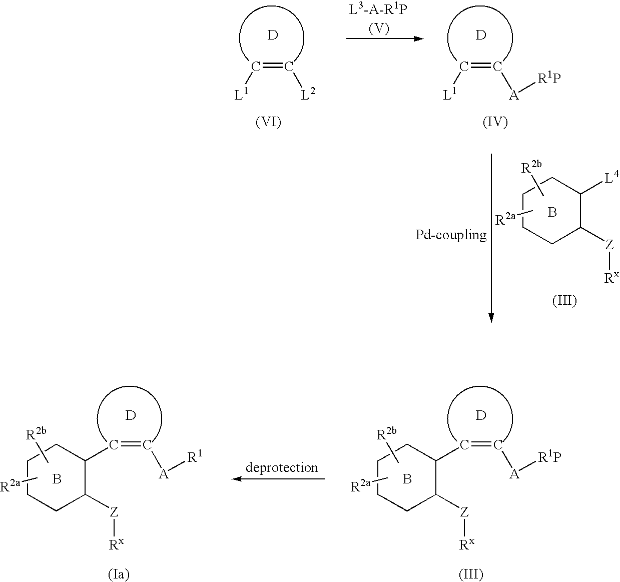

wherein L¹ and L² each represent a leaving group for example halo, or triflate; L³ and L⁴ each represent an activating group, for example boronic acid; P is an optional protecting group; D is an optionally substituted 5- or 6-membered heterocyclic ring containing one or two heteroatoms selected from N, S and O; and A, B, R¹, R^{2a}, R^{2b}, Z and R^{x} are as defined for compounds of formula (I). L: can be converted to L^{1a}, and L² can be converted to L^{2a} wherein L^{1a} and L^{2a} each represent an activating group for example a boronic acid, and in this situation L³ and L⁴ can each represent halo or triflate.

When R¹ is CO₂ examples of P include methyl, ethyl or optionally substituted benzyl esters.

Suitable reaction conditions for the deprotection of a compound of formula (II) include heating in aqueous ethanolic sodium hydroxide solution.

Suitable reaction conditions for the reaction of a compound of formula (VI) with a boronic acid of formula (V, L³ is —B(OH)₂), or a compound of formula (IV) with a boronic acid of formula (III, L⁴ is —B(OH)₂) include heating with tetrakis(triphenylphosphine)palladium (0) and an inorganic base, for example potassium carbonate, in a solvent, e.g. ethylene glycol dimethyl ether (DME), toluene and ethanol, preferably in a ratio of 1:1.

Accordingly the present invention also provides a process for the preparation of a compound of formula (Ia) or a derivative thereof:

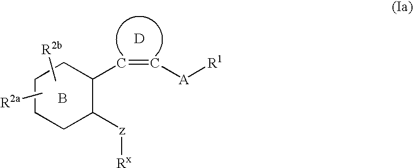

wherein:

A represents an optionally substituted aryl, or an optionally substituted 5 or 6-membered heterocycyl ring, or an optionally substituted bicyclic heterocyclyl group;
B represents a phenyl or pyridyl ring;
D represents an optionally substituted 5 or 6-membered heterocyclyl ring containing one or two heteroatoms selected from N, S and O;
Z represents O, S, SO, or SO₂;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, Coalkyl, 2H-tetrazol-5-yl-methyl, optionally substituted bicyclic heterocycle or optionally substituted heteroaryl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} represents optionally substituted CQ^{a}Q^{b}-heterocyclyl, optionally substituted C^{a}Q^{b}Q-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
Q^{a} and Q^{b} are each independently selected from hydrogen and CH₃;
wherein when A is a 6 membered ring the R¹ substituent and the D ring are attached to carbon atoms 1,2-, 1,3 or 1,4-relative to each other, and when A is a five-membered ring or bicyclic heterocyclyl group the R¹ substituent and the D ring are attached to substitutable carbon atoms 1,2- or 1,3-relative to each other;
comprising:
reacting a compound of formula (IV):

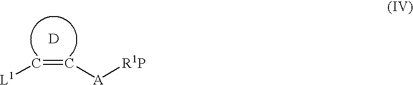

wherein A, D and R¹ are as hereinbefore defined above for a compound of formula (Ia), L¹ is a leaving group and P is an optional protecting group;
with a compound of formula (II):

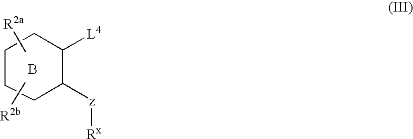

wherein R^{2a}, R^{2b}, B, Z, and R^{x} are as hereinbefore defined above for a compound of formula (I) and L⁴ is an activating group;
and where required converting:
one group A to another group A; and/or
one group R^{x} to another group R^{x};
and where required carrying out the following optional steps in any order
effecting deprotection; and/or
converting one group R¹ to another group R¹; and/or
forming a derivative of the compound of formula (Ia) so formed.

Alternatively compounds of formula (Ia) may be prepared according to the route described below:

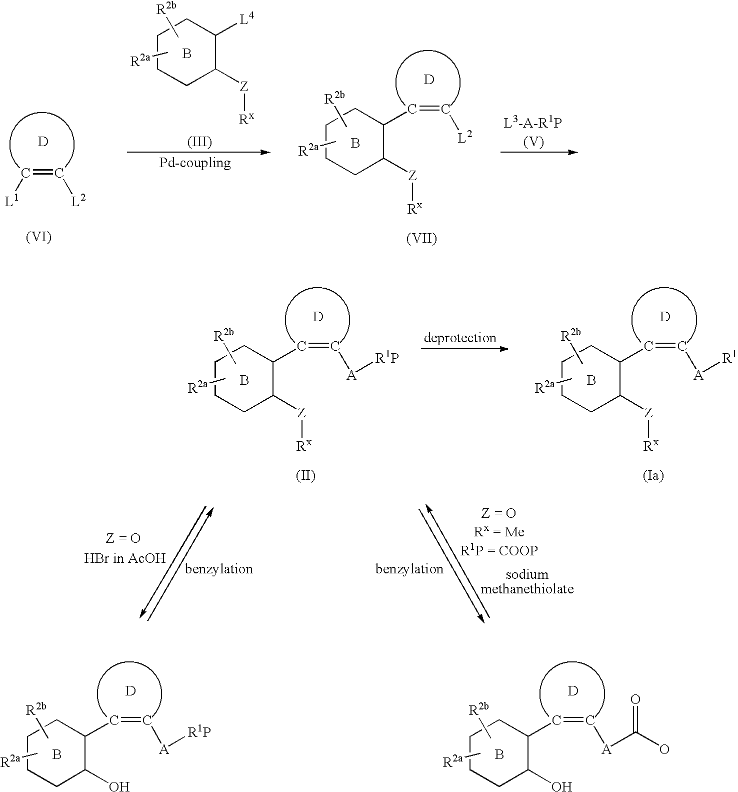

wherein L¹, L², L³, L⁴ and P are as defined above, and A, B, R^{1e}, R^{2b}, Z, and R^{x} are as defined for compounds of formula (I). L¹ can be converted to L₁ₐ, and L² can be converted to L^{2a} wherein L^{1a} and L^{2a} each represent an activating group for example a boronic acid, and in this situation L³ and L⁴ can each be halo or triflate.

Accordingly the present invention also provides a process for the preparation of a compound of formula (Ia) or a derivative thereof:

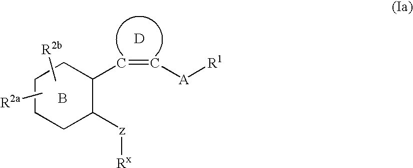

wherein:
A represents an optionally substituted aryl, or an optionally substituted 5- or 6-membered heterocyclyl ring, or an optionally substituted bicyclic heterocyclyl group;
B represents a phenyl or pyridyl ring;
D represents an optionally substituted 5- or 6-membered heterocyclyl ring containing one or two heteroatoms selected from N, S and O;
Z represents O, S, SO, or SO₂;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ,
wherein n is 0, 1 or 2; optionally substituted alkenyl; or optionally substituted alkynyl: or R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
Q^{a} and Q^{b} are each independently selected from hydrogen and CH₃;
wherein when A is a 6 membered ring the R¹ substituent and the D ring are attached to carbon atoms 1,2-, 1,3- or 1,4-relative to each other, and when A is a five-membered ring or bicyclic heterocyclyl group the R¹ substituent and the D ring are attached to substitutable carbon atoms 1,2- or 1,3-relative to each other;
comprising:
reacting a compound of formula (VII):

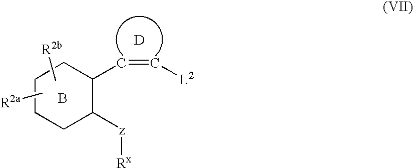

wherein R^{2a}, R^{2b}, B, D, R^{x} and R¹ are as hereinbefore defined above for a compound of formula (Ia), and L² is a leaving group;
with a compound of formula (V):

L³-A-R¹P (V)

wherein R¹, and A are as hereinbefore defined above for a compound of formula (I); L³ is an activating group and P is an optional protecting group;
and where required converting:
one group A to another group A; and/or
one group Rx to another group R^{x};
and where required carrying out the following optional steps in any order
effecting deprotection; and/or
converting one group R¹ to another group R¹; and/or
forming a derivative of the compound of formula (Ia) so formed.

Compounds of formula (Ib) which are compounds of formula (I) wherein the central D ring is 1H-pyrazolyl substituted by A on the 1-position and B on the 5 position may be prepared by the general route below.

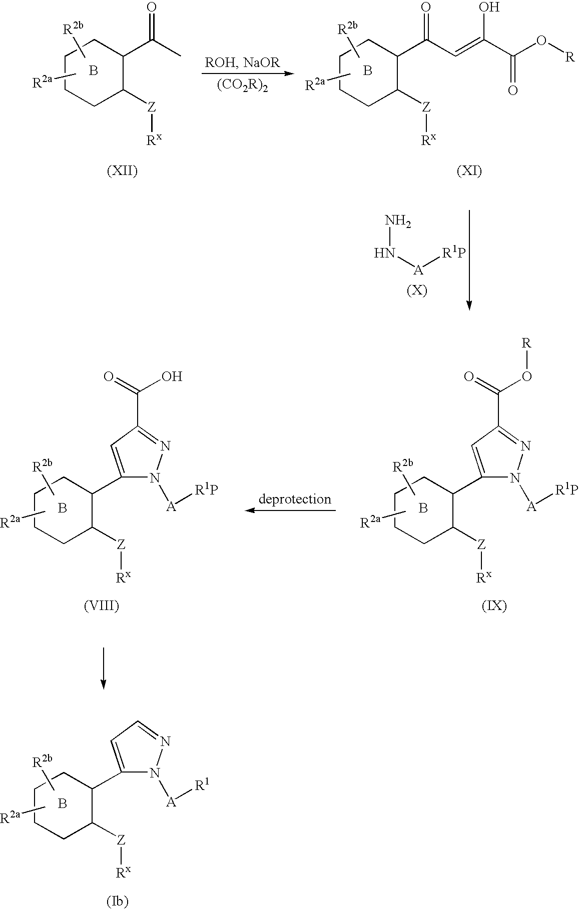

wherein A, B, R¹, R^{2a}, R^{2b}, Z, and RX are as hereinbefore defined for compounds of formula (I), R is an ester forming group such as C₁₋₄alkyl and P is an optional protecting group.

When R¹ is CO₂H, a suitable protecting group P is an ester forming group such as C₁₋₄alkyl or optionally substituted benzyl. Suitable reaction conditions for the deprotection of a compound of formula (II) include hydrolysis effected by e.g. heating in ethanolic sodium hydroxide solution, or hydrogenation.

Suitable conditions for the conversion of a compound of formula (VIII) to a compound of formula (Ib) include heating under nitrogen at 190-220° C.

Suitable reaction conditions for the reaction of a hydrazine (X) or a salt thereof with a compound of formula (XI) include heating at reflux in a solvent such as a C₁₋₄alcohol, e.g. methanol.

Suitable reaction conditions for the preparation of a compound of formula (XI) include reacting a methyl ketone of formula C(XI) with a dialkyl oxalate, e.g. dimethyl oxalate, in a C₁₋₄alcohol e.g. methanol, in the presence of a sodium alkoxide, e.g sodium methoxide.

Accordingly the present invention also provides a process for the preparation of a compound of formula (Ib) or a derivative thereof:

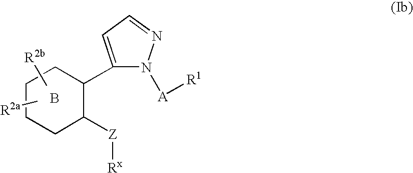

wherein:
A, B, Z, R¹, R^{2a}, R^{2b}, and R^{x} are as defined for compounds of formula (I) comprising:
reacting a compound of formula (XI):

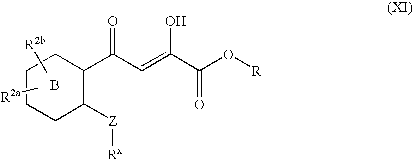

wherein R^{2a}, R^{2b}, B, R^{x}, and Z are as hereinbefore defined above for a compound of formula (Ib), and R is an ester forming group;
with a compound of formula (X):

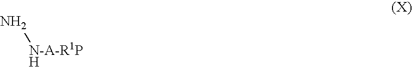

or a salt thereof, wherein R¹ and A are as hereinbefore defined above for a compound of formula (Ib); and P is an optional protecting group;
to give a compound of formula (IX):

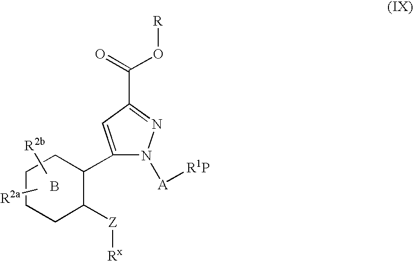

wherein R^{2a}, R^{2b}, A, B, Z, R^{x}, R¹, R and P are as hereinbefore defined
and, if necessary, effecting deprotection; and
effecting decarboxylation;
and if necessary converting one group R¹ to another group R¹; and/or forming a derivative of the compound of formula (Ib) so formed.

Compounds of formula (Ic) which are compounds of formula (I) wherein the central D ring is 1H-pyrrolyl substituted by A on the 2-position and B on the 1-position may be prepared by the general route below.

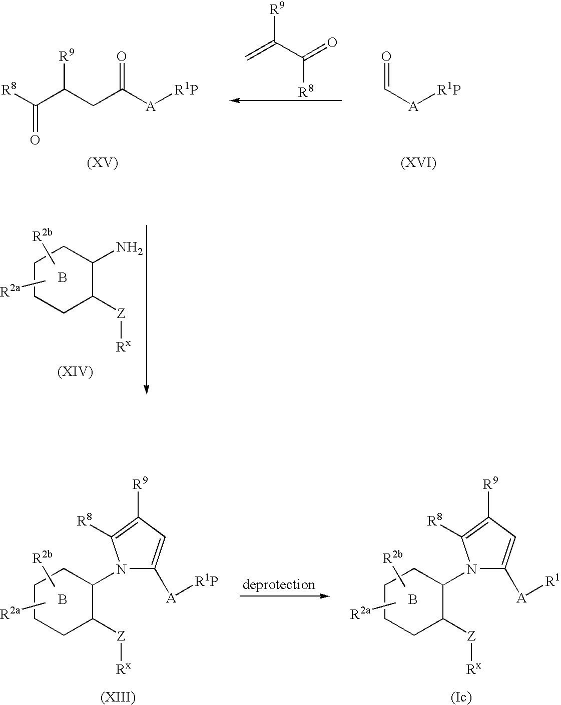

wherein A, B R¹, R^{2a}, R^{2b}, Z and R^{x} are as defined for compounds of formula (I), R⁸ and R⁹ independently selected from hydrogen, CF₃ or C₁₋₄alkyl, and P is an optional protecting group.

When R¹ is CO₂H, a suitable protecting group P is an ester forming group such as C₁₋₄alkyl or optionally substituted benzyl. Suitable reaction conditions for the deprotection of a compound of formula (II) include hydrolysis effected by e.g. heating in ethanolic sodium hydroxide solution, or hydrogenation.

Suitable reaction conditions for the reaction of a compound of formula (XIV) with a compound of formula (XV) to give a pyrrole of formula (XIII) include heating with an acid catalyst e.g. p-toluenesulfonic acid in a solvent such as toluene. Reviews of pyrrole synthesis can be found in e.g. A Triebs, Chem. Ber., 1957, 90, 79-84, E. Baltazzi et al, Chem. Rev., 1963, 63, 511, and R. A. Jones, Advances in Heterocyclyl Chemistry, 1970, 11, 383.

Suitable reaction conditions for the preparation of a compound of formula (XV) include reacting a compound of formula (XVI) with a suitable vinyl ketone e.g. methyl vinyl ketone in the presence of 3-ethyl-5-(2-hydroxyethyl)-4-methylthiazolium bromide and a base such as triethylamine in a solvent e.g. a C₁₋₄alcohol e.g. ethanol at reflux.

Accordingly the present invention also provides a process for the preparation of a compound of formula (Ic) or a derivative thereof:

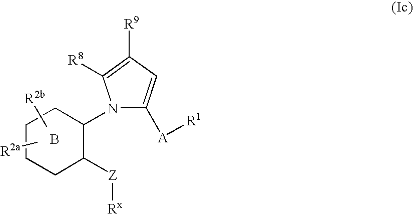

wherein:
wherein A, B, R¹, R^{2a}, R^{2b}, Z and R^{x} are as defined for compounds of formula (I), and R⁸ and R⁹ are independently selected from hydrogen, CF₃ or C₁₋₄alkyl;
comprising:
reacting a compound of formula (XV):

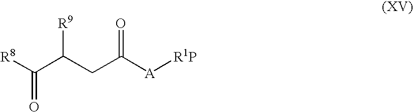

wherein A, R⁸, R⁹, and R¹ are as hereinbefore defined above for a compound of formula (Ic), and P is an optional protecting group;
with a compound of formula (XIV):

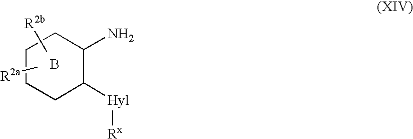

wherein R^{2a}, R^{2b}, B, Z, and R^{x} are hereinbefore defined above for a compound of formula (Ic); and P is an optional protecting group;
and where required converting:
one group A to another group A; and/or
one group R^{x} to another group R^{x},
and where required carrying out the following optional steps in any order:
effecting deprotection; and/or
converting one group R¹ to another group R¹; and/or
forming a derivative of the compound of formula (Ic) so formed.

A group R¹ may be converted to another group R¹ by use of conventional organic transformations known to those skilled in the art. For example R¹═CO₂H may be converted to an amide, e.g. CONHCQ^{a}Q^{b}aryl or CONHCQ^{a}Q^{b}heteroaryl wherein Q^{a} and Q^{b} are hydrogen or CH₃, by conventional methods for the preparation of amides as described in, for example, Richard Larock, Comprehensive Organic Transformations, 2nd edition, Wiley-VCH, ISBN 0-471-19031-4.

The preparation and reactions of boronic acids of formula (III) and formula (V) is reviewed in Suzuki et al, Synth. Commun., 1981, 11, 513; Martin et al, Acta. Chim. Scand., 1993, 47, 221; and Miyaura et al, Chem. Rev., 1995, 95, 2457. For example, 2-benzyloxy-5-chlorophenylboronic acid may be prepared from 2-benzyloxy-5-chloro-iodobenzene. 2-Benzyloxy-5-chloro-iodobenzene may be prepared from 4-chloro-2-iodoanisole by demethylation followed by benzylation according to known methods.

Certain substituents in any of the reaction intermediates and compounds of formula (I) may be converted to other substituents by conventional methods known to those skilled in the art. Examples of substituents which may be converted include one group RX to another group R^{x}; and one substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol or pyridinol is carried out using, for example, using acid e.g. HCl/dioxane or HBr/acetic acid, or using sodium methanethiolate. When R^{x} is methyl, cleavage of the ether to give the phenol is carried out using, for example, sodium methanethiolate. Cleavage of the ether to give a pyridinol is carried out in the presence of, for example, trifluoroacetic acid. Conversion to another R^{x} group, for example a substituted benzyl group, may be effected by reaction of the phenol or pyridinol with a suitable substituted benzyl bromide. The skilled person will appreciate that conversion of the protecting group P to another protecting group P may also occur under the reaction conditions used. When R^{x} is benzyl, cleavage of the ether to give the phenol or pyridinol may be carried out by hydrogenation according to known methods e.g. H₂—Pd/C or NH₄CO₂H—Pd/C. The resulting phenol or pyridinol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Compounds of the formula (III):

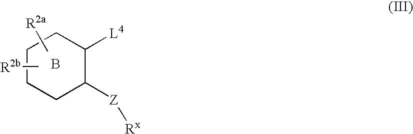

wherein L⁴ is as hereinbefore defined, R^{2a}, R^{2b}, Z, B and R^{x} and are as defined for compounds of formula (I) are commercially available, or may readily be prepared by methods known to those skilled in the art, for example from suitable commercially available pyridinols, anisoles or phenols using methods as described in the examples.

Intermediates of the formula (VI):

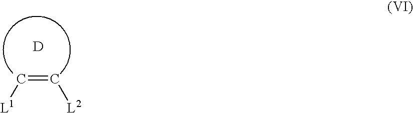

wherein L¹ and L² are as defined above, and D is as hereinbefore defined for compounds of formula (Ia) are commercially available or may be readily prepared according to known methods for the preparation of heterocycles. The preparation of heterocycles is reviewed in e.g. Comprehensive heterocyclic chemistry. The structure, reactions, synthesis and uses of heterocyclic compounds, A. R. Katritzky and C. W. Rees (Eds), vols 1-8, Pergamon Press, Oxford, 1984; Comprehensive organic chemistry II. A review of the literature 1982-1995, A. R. Katritzky, C. W. Rees, and E. F. V. Scriven (eds), vols 1-11, Pergamon Press, Oxford, 1996, and Heterocyclic Chemistry, 4th Edition, J. A. Joule and K. Mills, Blackwell Science, 2000.

Compounds of the formula (V):

L³-A-R¹P

wherein L³ and P are as defined above and R¹ and A are as hereinbefore defined for compounds of formula (I) are commercially available or may readily be prepared, for example, from suitable halobenzoic acid esters according to known methods, for example using methods as described in the examples.

Hydrazines of formula (X) are commercially available, or may be readily prepared by methods known to those skilled in the art for the preparation of hydrazines.

Intermediates of formula (XII) are commercially available, or may readily be prepared by standard transformations known to those skilled in the art, for example from suitable commercially available starting materials using methods as described in the examples. The preparation of ketones is reviewed in The Chemistry of the Carbonyl Group, S. Patai (Ed), Interscience, New York, 1966, and references cited therein.

Amines of formula (XIV) are commercially available, or may be prepared from commercial materials by standard transformations known to those skilled in the art for example using methods as described in the examples, e.g. from ortho-nitrophenols by reaction with R^{x}Br, followed by reduction of the nitro group to an amine group.

Aldehydes of formula (XVI) are commercially available, or may be made by standard methods as described, for example, in The Chemistry of the Carbonyl Group, S. Patai (Ed), Interscience, New York, 1966, and references cited therein.

Vinyl ketones of formula:

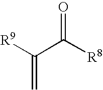

wherein R⁸ and R⁹ are as hereinbefore defined for compounds of formula (Ic) are commercially available or may be readily prepared according to known methods for the preparation of vinyl ketones. For example, F₃CCOCHCH₂═CH₂ may be prepared according to the method of M. Tordeux et al, J. Fluorine Chemistry, 1982, 20(3), 301-306. It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and they are therefore considered to be useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropatic pain, inflammatory pain, visceral pain, pain associated with cancer and fibromyalgia, pain associated with migraine, tension headache and cluster headaches, and pain associated with functional bowel disorders, non-cardiac chest pain and non-ulcer dispepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of this invention may also be useful in the treatment of visceral pain.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy, sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fanciers disease, farmers lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creuttfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhea) and colon cancer.

The compounds of formula (I) and pharmaceutically acceptable derivatives thereof are also useful in the treatment of overactive bladder and urge incontinence.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, or an inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT, agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B ssubtype; EP₄ receptor ligands; EP₂ receptor ligands; EP₃ receptor ligands; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day which may be administered as a single or divided dose, for example one to four times per day The dose range for adult human beings is generally from 8 to 2000 mg/day, such as from 20 to 1000 mg/day, preferably 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Bn (benzyl), Bu, Pr, Me, Et (butyl, propyl, methyl ethyl), DMSO (dimethyl sulfoxide), DCM (dichloromethane), DME (ethylene glycol dimethyl ether), DMF (N,N-dimethylformamide), EDC (1-(3-dimethylaminopropyl)-3-ethylcarbodiimide), EDTA (ethylenediamine tetraacetic acid), EtOAc (ethyl acetate), EtOH (ethanol), h (hour), HPLC (High pressure liquid chromatography), LCMS (Liquid chromatography/Mass spectroscopy), MDAP (Mass Directed Purification), MeOH (methanol), MeCN (acetonitrile), NMP (1-methyl-2-pyrrolidinone), NMR (Nuclear Magnetic Resonance (spectrum)), Ph (phenyl), pTSA (para-toluene sulphonic acid), SPE (Solid Phase Extraction), TBAF (tetrabutylammonium fluoride), THF (tetrahydrofuran), s, d, t, q, m, br (singlet, doublet, triplet, quartet, multiplet, broad.)

### LCMS

Column: 3.3 cm×4.6 mm ID, 3 um ABZ+PLUS
Flow Rate: 3 ml/min
Injection Volume: 5 μl
Temp: RT
UV Detection Range: 215 to 330 nm

| Solvents: | A: 0.1% Formic Add + 10 mMolar Ammonium Acetate. | | |
|---|---|---|---|
| | B: 95% Acetonitrile + 0.05% Formic Acid | | |
| Gradient: | Time | A % | B % |
| | 0.00 | 100 | 0 |
| | 0.70 | 100 | 0 |
| | 4.20 | 0 | 100 |
| | 5.30 | 0 | 100 |
| | 5.50 | 100 | 0 |

### Mass Directed Autopreparation

### Hardware:

Waters 600 gradient pump

Waters 2767 inject/collector

Waters Reagent Manager

Micromass ZMD mass spectrometer

Gilson Aspec—waste collector

Gilson 115 post-fraction UV detector

### Software:

Micromass Masslynx version 4.0

### Column

The column used is typically a Supelco LCABZ++ column whose dimensions are 20 mm internal diameter by 100 mm in length. The stationary phase particle size is 5 μm.

### Solvents:

A: Aqueous solvent=Water+0.1% Formic Acid

B: Organic solvent=MeCN:Water 95:5+0.05% Formic Acid

Make up solvent=MeOH:Water 80:20+50 mMol Ammonium Acetate

Needle rinse solvent=MeOH: Water DMSO 80:10:10

The method used depends on the analytical retention time of the compound of interest 15-minute runtime, which comprises a 10-minute gradient followed by a 5-minute column flush and re-equilibration step.

MDP 1.5-2.2=0-30% B

MDP 2.0-2.8=5-30% B

MDP 2.5-3.0=15-55% B

MDP 2.8-4.0=30-80% B

MDP 3.8-5.5=50-90% B

### Flow Rate:

flow rate 20 ml/min.

### Preparation of Intermediates

### [2-(Benzyloxy)-Phenyl]-carbamic Acid 2-trimethylsilanyl-ethyl Ester

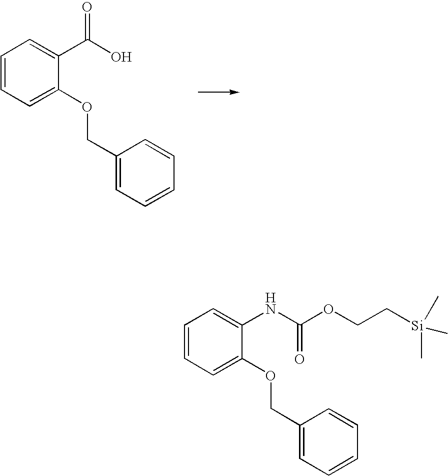

2-(Benzyloxy)benzoic acid (5.064 g, 22.2 mmol), diphenylphosphoryl azide (7.2 mL, 33.4 mmol. 1.5 eq) and triethylamine (4.6 mL, 33.4 mmol, 1.5 eq) were heated in toluene (44 mL, 0.5M) at 80° C. for 30 minutes. Trimethylsilyl ethanol (6.4 mL, 44.4 mmol) was added and heating was continued for 24 hours (complete after 1 hr). Upon cooling, the mixture was diluted with ethyl acetate and washed sequentially with 2M HCl and saturated sodium bicarbonate, dried (Na₂SO₄), filtered and concentrated. The residue was purified by chromatography on silica gel with iso-hexane containing ethyl acetate (1-3%) to yield the title compound (6.440 g, 85%) as a colourless oil.

¹H NMR (CDCl₃) δH, 0.00 (9H, s), 0.94-1.05 (2H, m), 4.13-4.27 (2H, m), 5.04 (2H, s), 6.81-6.97 (3H, m), 7.15 (1H, s), 7.25-7.41 (5H, m), 806 (1H, br).

### 2-(Benzyloxy)-aniline

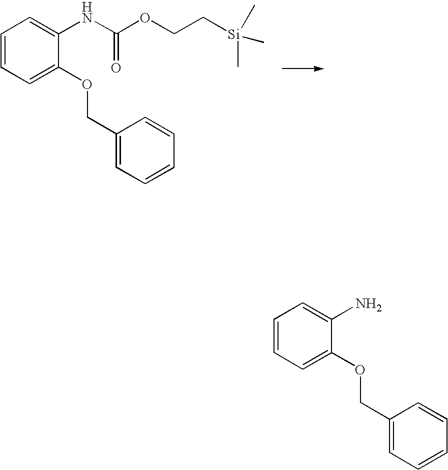

1M TBAF (tetrabutylammonium fluoride) in THF (tetrahydrofuran) (12 mL, 12.0 mmol) was added to [2-(benzyloxy)phenyl]-carbamic acid 2-trimethylsilanyl-ethyl ester (2.081 g, 6.1 mmol). The solution was stirred at room temperature for 4 hours, then more 1M TBAF in THF (6 ml, 6.0 mmol) was added and stirring continued for 2.5 hours. The mixture was diluted with diethyl ether and washed with water, dried (Na₂SO₄), filtered and evaporated to yield the title compound (1.208 g, 93%).

¹H NMR (CDCl₃) δH, 3.80 (2H, br), 5.07 (2H, s), 6.66-6.76 (2H, m), 6.77-6.89 (2H, m), 7.29-7.47 (5H, m).

### 2-(Benzyloxy)-5-chloro-nitrobenzene

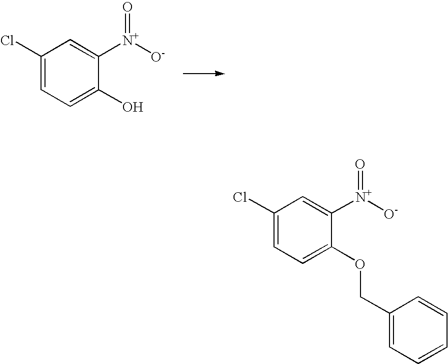

Benzyl bromide (10.7 mL, 119.8 mmol), 4-chloro-2-nitrophenol (10.348 g, 59.6 mmol) and potassium carbonate (16.535 g, mmol) were heated in DMF (N,N-dimethyl formamide) (60 mL) at 60° C. for 22 hours. Upon cooling, the mixture was diluted with diethyl ether and water. The layers were separated and the aqueous phase was extracted further with diethyl ether. The combined extracts were dried (Na₂SO₄), filtered and concentrated to yield the title compound (14.760 g, 94%).

¹H NMR (CDCl₃) δH: 5.26 (2H, s), 7.06 (1H, d, J=9 Hz), 7.30-7.48 (6H, m), 7.85 (1H, J=2 Hz).

### 2-(Benzyloxy)-5-chloro Aniline

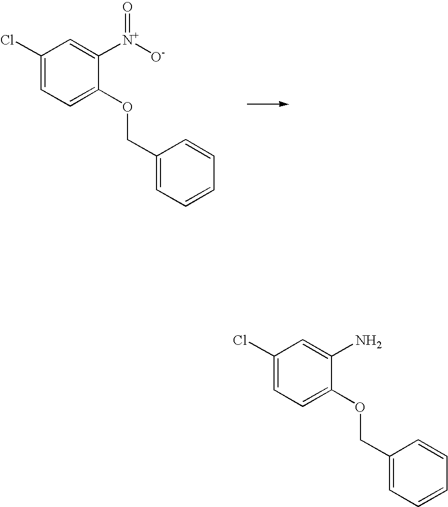

Zinc powder (13.200 g) was added slowly (heat evolved) to 2-(benzyloxy)-5-chloro-nitrobenzene (5.022 g, 19.1 mmol) in acetic acid (95 mL) at room temperature. The mixture was stirred overnight then filtered and evaporated. 2M sodium hydroxide and EtOAc were added to the residue. The layers were separated and the aqueous phase was extracted further with ethyl acetate. The combined extracts were dried (Na₂SO₄), filtered and evaporated to yield the title compound (4.420 g, 99%).

¹H NMR (CDCl₃) δH, 6.63 (1H, dd, J=2 Hz, J=9 Hz), 6.69 (1H, J=2 Hz), 6.73 (1H, d, J=9 Hz), 7.30-7.47 (5H, m).

### 2-(Benzyloxy)-5-bromo-nitrobenzene

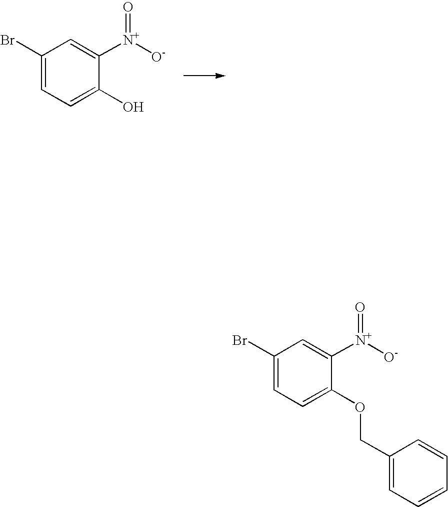

The title compound was prepared in an analogous fashion to 2-(benzyloxy 5-chloro-nitrobenzene, except 4-chloro-2-nitrophenol was replaced with 4-bromo-2-nitrophenol.

### 2-(Benzyloxy)-5-bromo-aniline

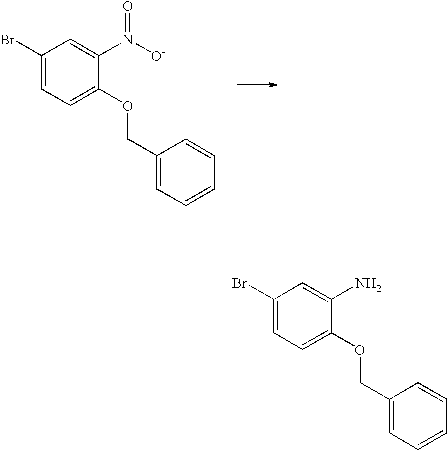

The title compound was prepared in an analogous fashion to 2-(benzyloxy)-5-chloro-aniline, except that 2-(benzyloxy)-5-chloro-nitrobenzene was replaced with 2-(benzyloxy)-5-bromo-nitrobenzene.

### 3-(4-Oxo-pentanoyl-benzoic Acid Methyl Ester

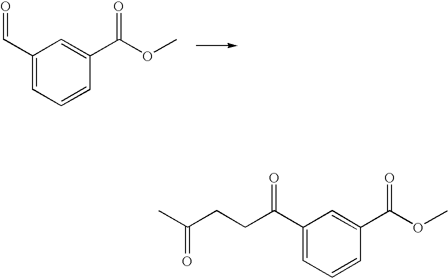

Methyl vinyl ketone (0.64 ml, 7.7 mmol, 1.2 eq), 3-formylbenzoate (1.048 g, 6.4 mmol), triethylamine (1.3 ml, 9.6 mmol, 1.5 eq) and 3-ethyl-5-(2-hydroxyethyl)-4-methylthiazolium bromide (355 mg, 1.4 mmol, 0.15 eq) were heated at reflux in ethanol (2.2 ml, 3M) for 4 hours. Upon cooling and dilution with ethyl acetate, the mixture was washed sequentially with saturated ammonium chloride and saturated sodium bicarbonate, dried (Na₂SO₄), filtered and evaporated. The residue was purified by chromatography on silica gel with iso-hexane containing a gradient of ethyl acetate (20-40%) to yield the title compound (1.324 g, 89%).

¹H NMR (CDCl₃) δH: 2.28 (3H, s), 2.92 (2H, t, J=6 Hz), 3.31 (2H, t, J=6 Hz), 3.96 (3H, s), 7.56 (1H, t, J=8 Hz), 8.18 (1H, dt, J=1 Hz, J=8 Hz), 8.24 (1H, dt, J=1 Hz, J=8 Hz), 8.63 (1H, s).

### Example 1

### 3-{1-[2-(Benzyloxy)phenyl]-5-methyl-1H-pyrrol-2-yl}-benzoic Acid

### a) 3-{1-[2-(Benzyloxy)phenyl]-5-methyl-1H-pyrrol-2-yl}-benzoic Acid Methyl Ester

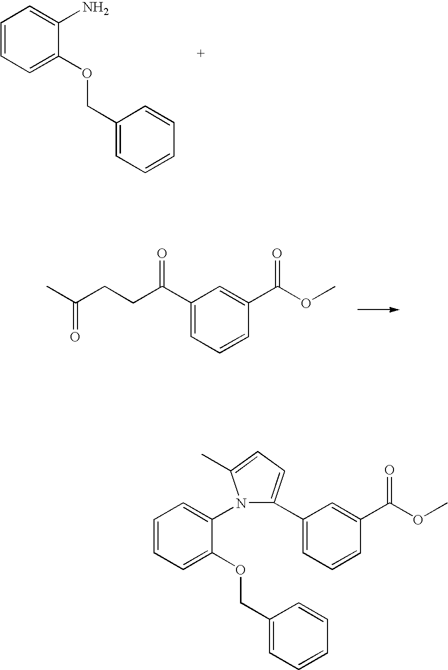

3-(4-Oxo-pentanoyl)benzoic acid methyl ester (250 mg, 1.1 mmol), 2-(benzyloxy)-aniline hydrochloride (240 mg, 1.0 mmol) and triethylamine (0.14 ml, 11.0 mmol) were heated in toluene (5 ml) in a reacti-vial at 120° C. for 24 hours. Upon cooling, the mixture was diluted with ethyl acetate and washed sequentially with 2M HCl and saturated sodium bicarbonate, dried (Na₂SO₄), filtered and concentrated. The residue was purified by chromatography on silica gel with iso-hexane containing an ethyl acetate gradient (5-10%) to yield the title compound (240 mg, 57%).

¹H NMR (CDCl₃) δH, 2.09 (3H, s), 3.81 (3H, s), 4.92 (1H, d, J=13 Hz), 5.03 (1H, d, J=13 Hz), 6.13 (1H, d, J=3.5 Hz), 6.45 (1H, d, J=3.5 Hz), 6.91-7.02 (2H, m), 7.03-7.35 (9H, m's excess), 7.72 (1H, d, J=6 Hz), 7.83 (1H, s).

### b) 3-{1-[2-(Benzyloxy-phenyl]-5-methyl-1H-pyrrol-2-yl}-benzoic Acid

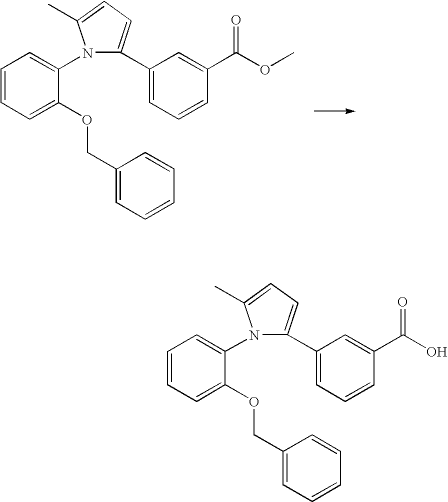

3-{1-[2-(Benzyloxy)-phenyl]-5-methyl-1H-pyrrol-2-yl}-benzoic acid methyl ester (129 mg, 0.3 mmol) was heated at 90° C. in a mixture of ethanol (2 ml) and 2M sodium hydroxide (1 ml) in a reacti-vial for 4 hours. The mixture was then cooled, diluted with ethyl acetate and washed with 2M HCl, dried (Na₂SO₄), filtered and concentrated to yield the title compound (120 mg, 96%). LC/MS Rt=3.74 min [MH+] 384, [MH−] 382.

### Example 2

### 3-{1-[2-(Benzyloxy)-5-chloro-phenyl]-5-methyl-1H-pyrrol-2-yl}-benzoic Acid

### a) 3-{1-[2-(Benzyloxy)-5-chloro-phenyl]-5-methyl-1H-pyrrol-2-yl}-benzoic Acid Methyl Ester

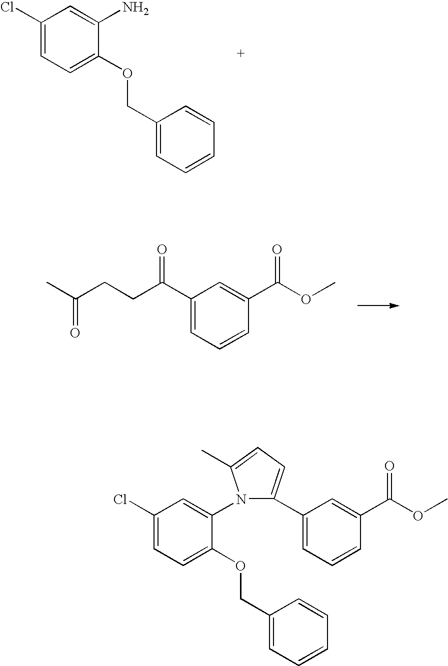

3-(4-Oxo-pentanoyl)benzoic acid methyl ester (206 mg, 0.9 mmol), (2-benzyloxy)-5-chloro-aniline (254 mg, 1.1 mmol) and pTSA (22 mg, cat.) were heated at reflux in toluene (8.8 ml) for 3.5 hours. Upon cooling, the mixture was diluted with ethyl acetate and washed sequentially with 2M HCl and saturated sodium bicarbonate, dried (Na₂SO₄), filtered and concentrated. The residue was purified by chromatography on silica gel with iso-hexane containing an ethyl acetate gradient (3-5%) to yield the title compound (280 mg, 74%).

¹H NMR (CDCl₃) δH, 2.09 (3H, s), 3.83 (3H, s), 4.88 (1H, d, J=12.5 Hz), 5.00 (1H, d, J=12.5 Hz), 6.12 (1H, d, J=3 Hz), 6.43 (1H, d, J=3 Hz), 6.87 (1H, d, J=9 Hz), 7.02-7.10 (2H, m), 7.16-7.29 (7H, m's excess), 7.76 (1H, d, J=7 Hz), 7.83 (1H, s).

LC/MS Rt=4.06 min [MH+] 432, 434.

### b) 3-{1-[2-(Benzyloxy)-5-chloro-phenyl]-5-methyl-1H-pyrrol-2-yl}-benzoic Acid

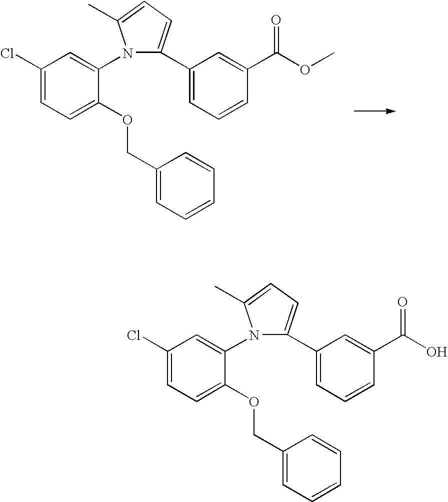

3-{1-[2-(Benzyloxy)-5-chloro-phenyl]-5-methyl-1H-pyrrol-2-yl}-benzoic acid methyl ester (280 mg, 0.7 mmol) was heated at reflux in a mixture of ethanol (6 ml) and 2M sodium hydroxide (3 ml) for 2.5 hours. The mixture was then cooled, diluted with ethyl acetate and washed with 2M HCl, dried (Na₂SO₄), filtered and concentrated to yield the title compound (268 mg, 99%).

¹H NMR (CDCl₃) δH: 2.09 (3H, s), 4.90 (1H, d, J=13 Hz), 5.01 (1H, d, J=13 Hz), 6.13 (1H, d, J=4 Hz), 6.44 (1H, d, J=4 Hz), 6.88 (1H, d, J=9 Hz), 7.05-7.11 (2H, m), 7.18 (1H, d, J=3 Hz), 7.02-7.33 (6H, m's excess), 7.83 (1H, d, J=8 Hz), 7.90 (1H, s).

LC/MS Rt=3.88 [MH+] 418 & 420, [MH−] 416 & 418.

### Example 3

### 3-{1-[2-(Benzyloxy)-5-bromo-phenyl]-5-methyl-1H-pyrrol-2-yl}-benzoic Acid

### a) 3-{1-[2-(Benzyloxy-5-bromo-phenyl]-5-methyl-1H-pyrrol-2-yl}-benzoic Acid Methyl Ester

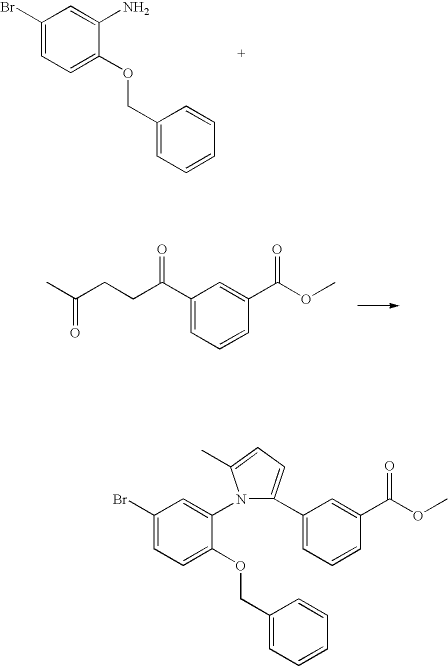

The title compound was prepared in an analogous fashion to 3-{1-[2-(benzyloxy)-5-chloro-phenyl]-5-methyl-1H-pyrrol-2-yl}-benzoic acid methyl ester except that 2-benzyloxy)-5-chloroaniline was replaced by 2-(benzyloxy)-5-bromo aniline and the mixture was heated at reflux for 2 hours.

### b) 3-{1-[2-(Benzyloxy-5-bromo-phenyl]-5-methyl-1H-pyrrol-2-yl}-benzoic Acid

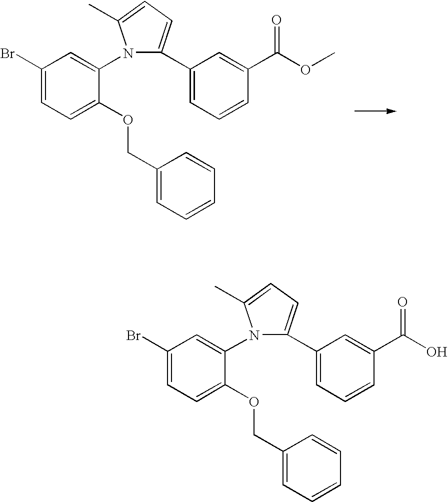

The title compound was prepared in an analogous fashion to 3-{1-[2-(benzyloxy 5-chloro-phenyl]-5-methyl 1H-pyrrol-2-yl}-benzoic acid except that 3-{1-[2-(benzyloxy)-5-chloro-phenyl]-5-methyl-1H-pyrrol-2-yl}benzoic acid methyl ester was replaced by 3-{1-[2-(Benzyloxy)-5-bromo-phenyl]-5-methyl-1H-pyrrol-2-yl}-benzoic acid methyl ester.

LC/MS Rt=3.90 [MH+] 462, [MH−] 460.

### Example 4

### 3-{5-[2-(Benzyloxy)-phenyl]-1H-pyrazol-1-yl}-benzoic Acid

### a) 4-[2-(Benzyloxy)-phenyl]-2,4-dioxobutyric Acid Methyl Ester

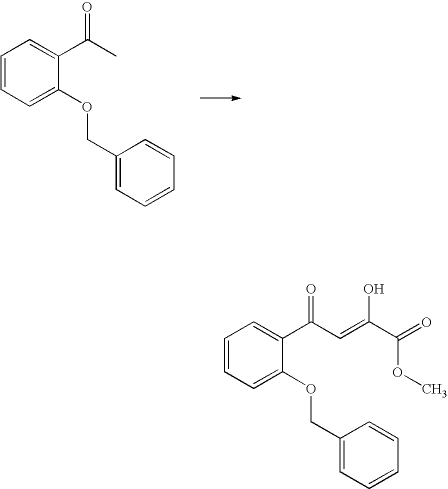

2-Benzyloxyacetophenone (2.230 g, 9.87 mmol) was stirred at room temperature in MeOH (20 mL) with sodium methoxide (1.074 g) and dimethyl oxalate (2.379 g). After 24 hours the mixture was quenched with saturated ammonium chloride and extracted with ethyl acetate. The extracts were dried (Na₂SO₄), filtered and evaporated. The residue was chromatographed on silica gel with hexane containing ethyl acetate (5-20%) to yield the title compound, 2.490 g (81%).

### b) 3-{3-[(Methoxy)carbonyl]-5-[2-(benzyloxy)phenyl]-1H-pyrazol-1-yl}-benzoic Acid

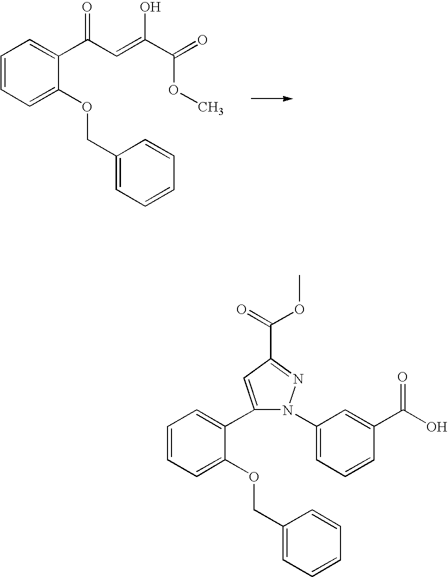

4-[2-(Benzyloxy)-phenyl]-2,4-dioxo-butyric acid methyl ester (312 mg, 1 mmol) and 3-hydrazinobenzoic acid (167 mg, 1.1 mmol) were dissolved in methanol (5 ml) and the solution heated to reflux for 1 hour. After cooling, the solvent was evaporated and the residue partitioned between dichloromethane and water. The organic layer was dried (MgSO₄), evaporated to dryness and dissolved in diethyl ether. A cream solid crystallised and was filtered, washed with diethyl ether and dried in vacuo to give the title compound (330 mg).

LC/MS Rt=−3.46 [MH+] 428. (contains ca. 20% [MH⁺] 446).

### c) 3-{3-Carboxy-5-[2-(benzyloxy)-phenyl]-1H-pyrazol-1-yl}-benzoic Acid

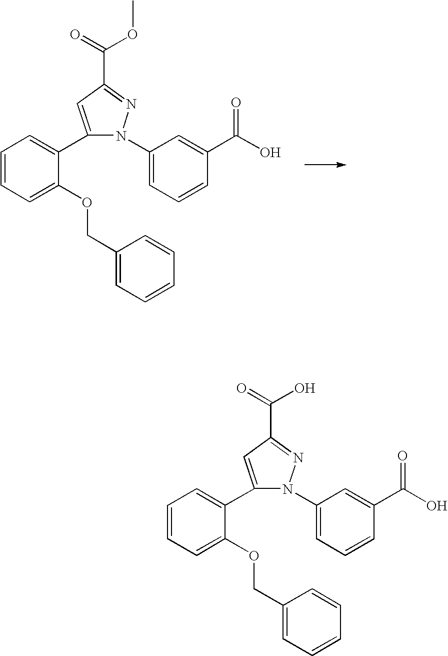

3-{3-[(Methyloxy)carbonyl]-5-[2-(benzyloxy)-phenyl]-1H-pyrazol-1-yl}-benzoic acid (330 mg, 0.77 mmol) was dissolved in ethanol (2 ml) and 2M sodium hydroxide solution (1 ml) added. The mixture was heated to reflux for 2 hours. After cooling, the ethanol was evaporated and the residue diluted with water and extracted with diethyl ether. The aqueous layer was acidified with 2M hydrochloric acid solution and extracted with diethyl ether (×2). The combined organic layers were washed with water, dried (MgSO₄) and evaporated. The residue was triturated with diethyl ether and the title compound filtered and dried in vacuo (205 mg). LC/MS Rt=3.65 [MH+] 415.

### d) 3-{5-[2-(Benzyloxy)-phenyl]-1H-pyrazol-1-yl}-benzoic Acid

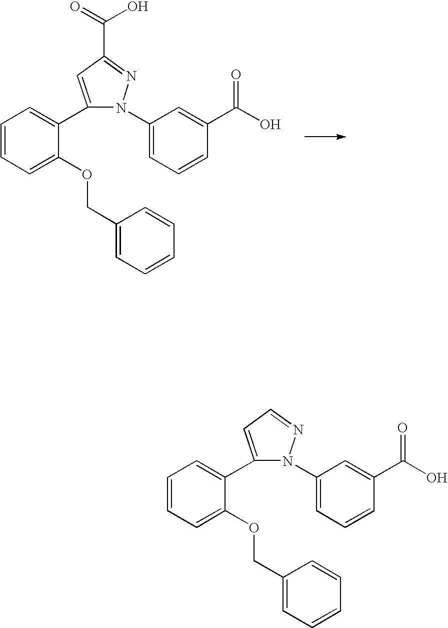

3-{3-Carboxy-5-[2-(benzyloxy)-phenyl]-1H-pyrazol-1-yl}-benzoic acid (50 mg, 0.121 mmol) was heated under nitrogen at 190-220° C. for 15 minutes. The product was purified by preparative HPLC and trituration with isohexane containing a trace of dichloromethane to give the title compound as a cream solid. (12.5 mg)

¹H NMR (CDCl₃) δH: 4.75 (2H, s), 6.51 (1H, s), 6.86 (1H, d, J=8 Hz), 6.96-6.98 (2H, m), 7.00-7.04 (1H, t, J=5 Hz), 7.23-7.41 (7H, m), 7.89 (1H, s), 7.93 (1H, d, J=3 Hz), 8.04 (1H, s) LC/MS Rt=3.45 [MH+] 371.

### Example 5

### 3-{5-[2-(Benzyloxy)-5-chloro phenyl]-1H-pyrazol-1-yl}benzoic Acid

### a) 4-[2-(Benzyloxy)-5-chloro-phenyl]-2,4-dioxobutyric Acid Methyl Ester

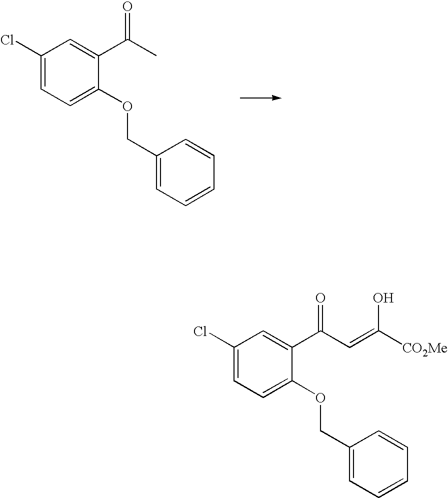

The title compound was prepared in a similar manner to 4-[2-(benzyloxy)phenyl]-2,4-dioxobutyric acid methyl ester starting from 5-chloro-2-benzyloxyacetophenone.

LC/MS Rt 3.79 min, [MH−]345, 347.

### b) 3-{3-[(Methyloxy)carbonyl]-5-[2-(benzyloxy 5-chloro-phenyl]-1H-pyrazol-1-yl}-benzoic Acid

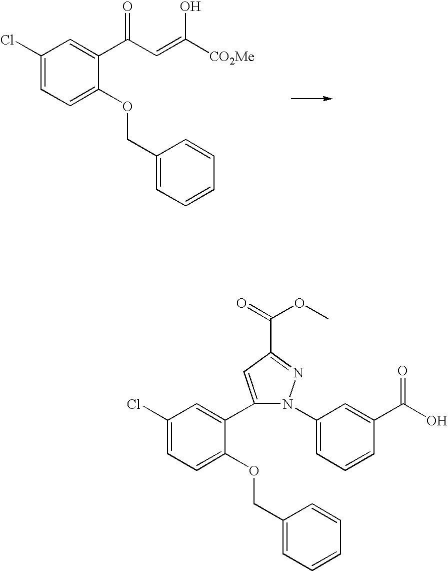

The title compound was prepared in a similar manner to 3-{3-[(methyloxy)carbonyl]-5-[2-(benzyloxy)-phenyl]-1H-pyrazol-1-yl}-benzoic acid staring from 4-[2-(benzyloxy)-5-chloro phenyl]-2,4-dioxobutyric acid methyl ester. LC/MS Rt 3.63 min, [MH+] 463, 465.

### c) 3-{3-Carboxy-5-[2-(benzyloxy)-5-chloro-phenyl]-1H-pyrazol-1-yl}-benzoic Acid

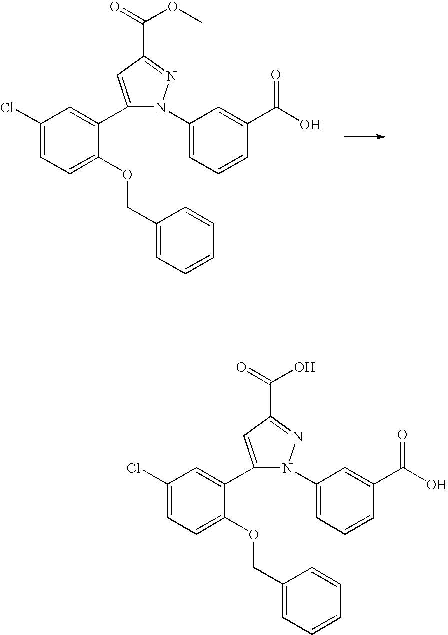

The title compound was prepared in a similar manner to 3-{3-carboxy-5-[2-(benzyloxy)phenyl]-1H-pyrazol-yl}-benzoic acid starting from 3-{3-[(methyloxy)carbonyl]-5-[2-(benzyloxy)-5-chloro-phenyl]-1H-pyrazol 1-yl}-benzoic acid.

LC/MS Rt 4.03 min, [MH+] 449, 451.

### d) 3-{5-[2-(Benzyloxy)-5-chloro-phenyl]-1H-pyrazol-1-yl}-benzoic Acid

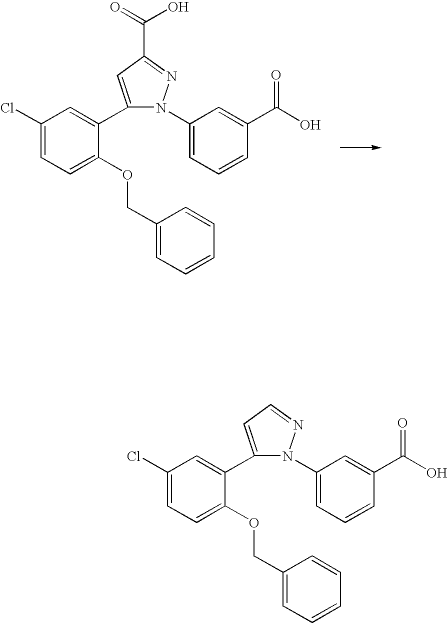

The title compound was prepared in a similar manner to 3-{5-[2-(benzyloxy)-phenyl]-1H-pyrazol-1-yl}-benzoic acid starting from 3-{3-carboxy-5-[2-(benzyloxy)-5-chlorophenyl]-1H-pyrazol-1-yl}benzoic acid.

¹H NMR (CDCl₃) δH: 4.70 (2H, s), 6.50 (1H, s), 6.77 (1H, d, J=5 Hz), 6.92 (2H, m), 7.23-7.39 (7H, m), 7.83 (1H, s), 7.96 (1H, d, J=3 Hz), 8.03 (1H, s).

### Example 6

### 3-{3-[2-(Benzyloxy)-5-chloro-phenyl]-pyrazin-2-yl}-benzoic Acid

### a) 2-[2-(Benzyloxy)-5-chloro]-3-chloro-pyrazine

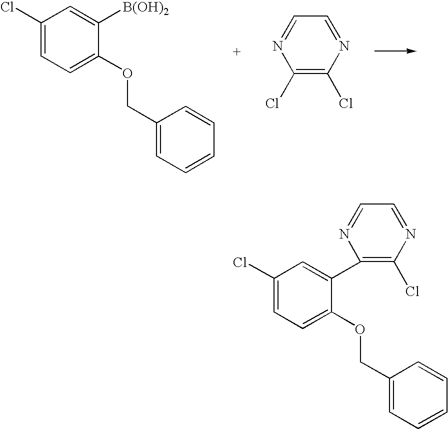

2,3-Dichloropyrazine (255 mg, 1.71 mmol), 2-(benzyloxy)-5-chloro-phenylboronic acid (450 mg, 1.71 mmol) and sodium carbonate (139 mg) were stirred in DME (ethylene glycol dimethyl ether)-water (1:1, 10 mL) under nitrogen for 20 minutes, then tetrakis(triphenylphoshine)palladium(0) (100 mg) was added and the mixture heated at reflux for 20 hours. Upon cooling, saturated ammonium chloride was added and the resultant mixture was extracted with DCM (×2). The combined extracts were dried (MgSO₄), filtered and evaporated. The residue was chromatographed on silica gel with cyclohexane containing ethyl acetate (5-100%) to yield the title compound (448 mg, 80%).

LC/MS Rₜ=3.47 min, [MH+] 331.

### b) 3-{3-[2-(Benzyloxy)-5-chloro-phenyl]-pyrazin-2-yl}-benzoic Acid

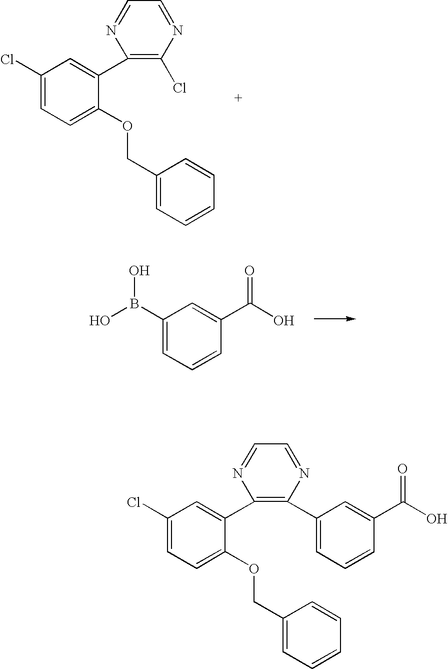

2-[2-(Benzyloxy)-5-chloro-phenyl]-3-chloro-pyrazine (200 mg, 0.61 mmol), 3-carboxyphenyl boronic acid (101 mg, 0.61 mmol) and sodium carbonate (51 mg) were stirred in DME-water (1:1, 4 mL) for 30 minutes then tetrakis(triphenylphosphine)palladium(0) (40 mg) was added and the mixture heated at reflux for 18 hours. Upon cooling to room temperature saturated ammonium chloride was added and the resultant mixture was extracted with DCM (×2). The combined extracts were dried (MgSO₄), filtered and evaporated. The residue was purified by MDAP to give the title compound (10 mg).

LC/MS Rt 3.55 min, [MH+] 417.

### Example 7

### 3-{4-[2-(Benzyloxy)-5-Chloro-phenyl]-2-oxo-2,5-dihydro-furan-3-yl}-benzoic Acid

### a) 3-Bromo-4-[2-(benzyloxy)-5-chloro-phenyl]-2(5H)-furanone

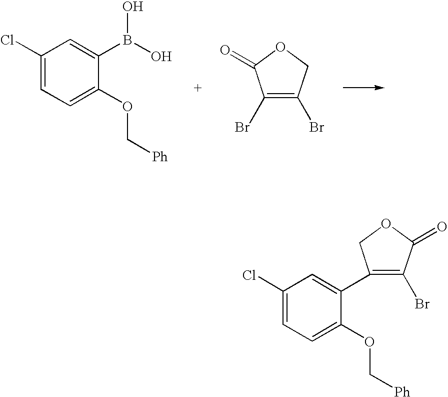

2-(Benzyloxy)-5-chloro-phenylboronic acid (1.58 g, 6 mmol) and 3,4-dibromo-2(5H) furanone (1.21 g, 5 mmol) were dissolved in tetrahydrofuran (50 ml) under nitrogen and bis(acetonitrile)dichloropalladium(II) (130 mg, 0.5 mmol), triphenylarsine (310 mg, 1 mmol) and silver (II) oxide (3.48 g, 15 mmol) added. The mixture was stirred and heated to 50° C. for 16 hours. Ethyl acetate (125 ml) was added and the mixture filtered through a pad of Kieselguhr. The filtrate was washed with water (×2), dried (MgSO₄) and evaporated. The residue was purified by chromatography on silica gel, eluting with 5-20% ethyl acetate in isohexane. The product was triturated with diethyl ether/isohexane and the solid filtered and dried in vacuo to give the title compound. (738 mg).

¹H NMR (CDCl₃) δH: 5.09 (2H, s), 5.16 (2H, s), 7.00 (1H, d, J=9 Hz), 7.34-7.42 (6H, m), 7.79 (1H, d, J=2.5 Hz).

### b) 3-{4-[2-(Benzyloxy)-5-chloro-phenyl]-2-oxo-2,5-dihydro-furan-3-yl}-benzoic Acid

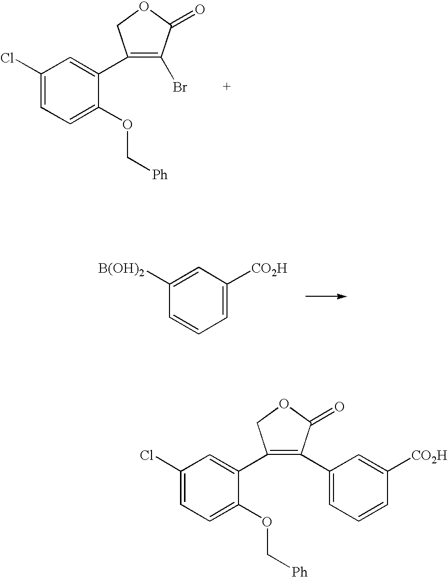

3-Bromo-4-[2-(benzyloxy)-5-chloro-phenyl}-2(5H)furanone (95 mg, 0.25 mmol) and 3-(carboxy)phenylboronic acid (50 mg, 0.3 mmol) were dissolved in tetrahydrofuran (4 ml) under nitrogen and bis(acetonitrile)dichloropalladium(II) (7 mg, 0.025 mmol), triphenylarsine (16 mg, 0.05 mmol) and silver (II) oxide (174 mg, 0.75 mmol) added. The mixture was stirred and heated to 80° C. for 8 hours then left stirring at room temperature for 72 hours. Ethyl acetate (20 ml) was added and the mixture filtered through a pad of Kieselguhr. The filtrate was washed with water (×2), dried (MgSO₄) and evaporated. The residue was purified by chromatography on silica gel, eluting with 5-20% ethyl acetate in isohexane then 80% ethyl acetate in isohexane. The product was further purified by preparative HPLC to give the title compound (10.5 mg). LC/MS Rt 3.52 min, [MH⁻] 419.

### Example 8

### 3-{3-[2-(Benzyloxy)-5-chloro-phenyl]-2-oxo-2,5-dihydro-furan-4-yl}-benzoic Acid

### a) 3-(3-Bromo-2-oxo-2,5-dihydrofuran-4-yl)-benzoic Acid tert-butyl Ester

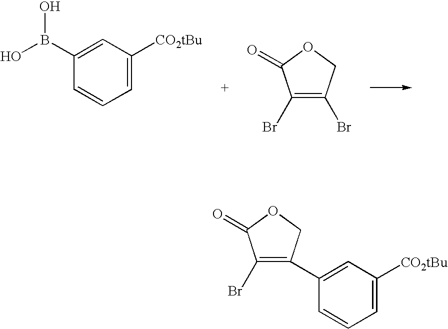

3-(tert-Butoxycarbonyl)-phenylboronic acid (133 mg, 0.6 mmol) and 3,4-dibromo-2(5H) furanone (121 mg, 0.5 mmol) were dissolved in tetrahydrofuran (5 ml) under nitrogen and bis(acetonitrile)dichloropalladium(II) (13 mg, 0.05 mmol), triphenylarsine (31 mg, 0.1 mmol) ans silver (II) oxide (348 mg, 1.5 mmol) added. The mixture was stirred and heated to 50° C. for 4 hours. Ethyl acetate (20 ml) was added and the mixture filtered through a pad of Kieselguhr. The filtrate was washed with water (×2), dried (MgSO₄) and evaporated. The residue was purified by chromatography on silica gel, eluting with 5-20% ethyl acetate in isohexane to give the title compound. (60 mg). LC/MS Rt 3.41 min, [MH⁺] 339, 341.

### b) 3-{3-[2-Benzyloxy)-5-chloro-phenyl]-2-oxo-2,5-dihydro furan-4-yl}-benzoic Acid tert-butyl Ester

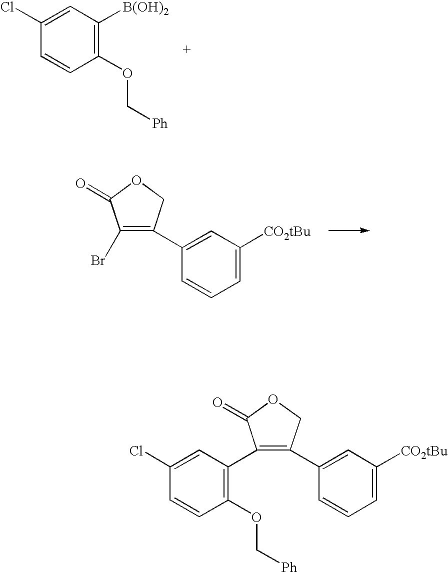

3-(3-bromo-2-oxo-2,5-dihydrofuran-4-yl)-benzoic acid tert-butyl ester (60 mg, 0.177 mmol) and 2-(benzyloxy)-5-chlorophenylboronic acid (56 mg, 0.212 mmol) were dissolved in tetrahydrofuran (2 ml) under nitrogen and bis(acetonitrile)dichloropalladium(II) (5 mg, 0.0177 mmol), triphenylarsine (9 mg, 0.035 mmol) and silver (II) oxide (103 mg, 0.531 mmol) added. The mixture was stirred and heated to 60° C. for 16 hours. The mixture was then filtered through a pad of Kieselguhr and washed with ethyl acetate. The filtrate was evaporated. The residue was dissolved in tetrahydrofuran (2 ml) under nitrogen and 2-(benzyloxy)-5-chlorophenylboronic acid (28 mg, 0.106 mmol). Bis(acetonitrile)dichloropalladium(II) (2.5 mg, 0.0088 mmol), triphenylarsine (4.5 mg, 0.018 mmol) and silver (II) oxide (52 mg, 0.265 mmol) added. The mixture was stirred and heated to 60° C. for 16 hours. Ethyl acetate (10 ml) was added and the mixture filtered through a pad of Kieselguhr The filtrate was washed with water (×2), dried (MgSO₄) and evaporated. The residue was purified by chromatography on silica gel, eluting with 5-15% ethyl acetate in isohexane to give the title compound. (37 mg).

LC/MS Rt 3.90 min, [MH⁻] 475, 477.

### c) 3-{3-[2-(Benzyloxy)-5-chloro-phenyl]-2-oxo-2.5-dihydro-furan-4-yl}-benzoic Acid

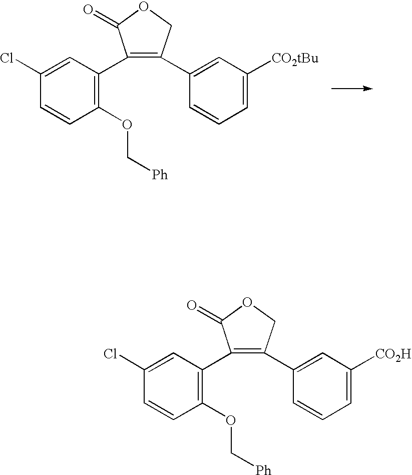

3-{3-[2-(Benzyloxy)-5-chloro-phenyl]-2-oxo-2,5-dihydro-furan-4-yl}-benzoic acid tert-butyl ester (37 mg, 0.078 mmol) was dissolved in dichloromethane (3 ml) and trifluoroacetic acid (1 ml) added. The mixture was stirred at room temperature for 16 hours. The mixture was evaporated and the residue re-evaporated from toluene. The crude product was purified by preparative HPLC and triturated with diethyl ether. Filtration gave the title compound as an off-white solid. (10 mg). LC/MS Rt 3.45 min, [MH⁻] 419, 421.

### Preparation of Intermediates

### 2-(Benzyloxy)-5-chloro-iodobenzene

4-Chloro-2-iodophenol (57 g. 0.22 mol) was dissolved in acetonitrile (500 ml), caesium carbonate (72.6 g, 0.22 mol.) was added slowly giving rise to an exotherm (19-24° C.) over 30 minutes. The reaction mixture was then kept at 24° C. for a further 5 hours. The reaction mixture was then stirred at 40° C. for 4 hours, then stirred at room temperature over night. The reaction mixture was filtered and evaporated down to a pink/brown solid. After trituration with water (200 ml) the suspension was filtered and recrystallised from hexane (200 ml) giving the title compound 50.2 g, 65% yield. A second crop gave a further 22.7 g. Total yield after drying 88%.

### 2-(Benzyloxy)-5-chloro-phenylboronic Acid

2-(Benzyloxy)-5-chloro-iodobenzene (5 g 0.0145 mol) in diethyl ether/tetrahydrofuran (100:30) was cooled to −100° C. n-Butyl lithium, 1.6M solution in hexanes (10 mL, 0.016 mol) was added dropwise over 15 min under nitrogen. The reaction mixture was then allowed to rise to −70° C. for 1 h. Triethlborate (9 mL, 0.03 mol) was added dropwise under nitrogen. The cooling bath was then removed and the reaction mixture was stirred at room temperature overnight. The reaction mixture was then quenched with 2N hydrochloric acid (40 mL) and stirred vigorously at room temperature for 1 h. The product was then extracted with ethyl acetate, dried over magnesium sulphate and evaporated down to an oil. Purification was carried out on silica gel with diethyl ether/iso-hexane (50:50) to give the title compound (2.8 g, 74% yield).

### Ethyl 3-(3-bromopyridin-4-yl)benzoate

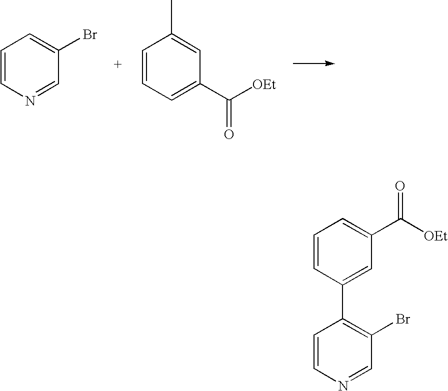

1.6M butyllithium in hexanes (20.2 ml, 32.32 mmol) was added over 5 minutes to a solution of diisopropylamine (3.27 g, 32.35 mmol) in anhydrous THF (70 ml) at −78° C. under nitrogen and stirred for 10 minutes then allowed to warm to room temperature. This mixture was then added to a solution of 3-bromopyridine (4.26 g, 27 mmol) in THF (50 ml) at −95° C. over 15 minutes. After stirring for 25 minutes 0.5M zinc chloride solution in THF (53.9 ml, 26.95 mmol) was added over 15 minutes and the mixture allowed to warm to room temperature. Ethyl 3-iodobenzoate (2.76 g, 10 mmol) and tetrakis(triphenylphosphine)palladium(0) (570 mg, 0.5 mmol) were added and the mixture refluxed for 2.5 hours then cooled and evaporated to dryness. The residue was dissolved in ether 1M sodium hydroxide solution and the organic phase was dried (magnesium sulphate), evaporated and purified by chromatography on silica eluting with ethyl acetate/iso-hexane (1:4) then rechromatographed eluting with methanol/dichloromethane (1:199). Recrystallisation from iso-hexane gave the title compound (1.36 g) as a white solid. LC/MS t=3.26, [MH+] 307.9

### 3-{3-[2-(Benzyloxy)-5-chloro-phenyl]-pyridin-4-yl}-benzoic Acid Ethyl Ester

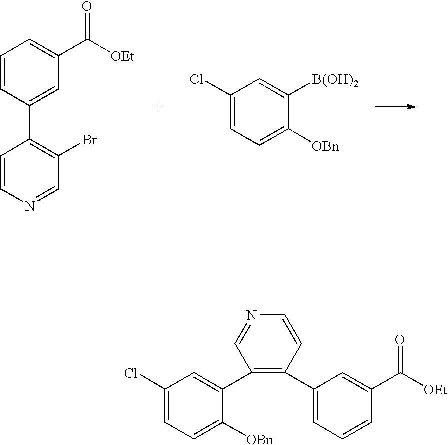

A mixture of ethyl 3-(3-bromopyridin-4-yl)benzoate (156 mg, 0.51 mmol), 2-(benzyloxy-5-chlorophenylboronic acid (157 mg, 0.6 mmol), potassium carbonate (568 mg, 4.11 mmol) and tetrakis(triphenylphosphine)palladium(0) (66 mg, 0.057 mmol) was stirred and heated at 90° C. in 1:1 toluene/ethanol (5 ml) for 16 hours then cooled to room temperature and diluted with ether/water. The organic phase was dried (magnesium sulphate), evaporated and purified by chromatography on silica eluting with ethyl acetate/iso-hexane (1:3) to give the title compound (179 mg) as a colourless gum.

LC/MS t=3.79, [MH+] 444.0, 446.0.

### 3-{3-[2-(Benzyloxy)-phenyl]-pyridin-4-yl}-benzoic Acid Ethyl Ester

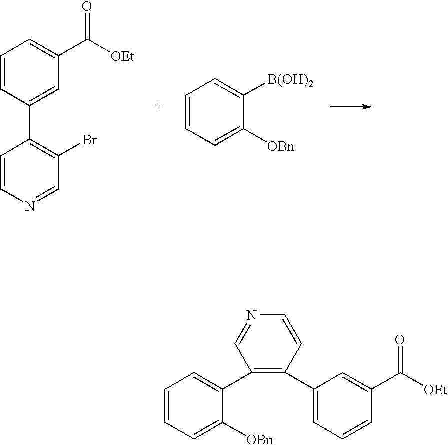

The title compound was prepared using an analogous procedure to that used for the preparation of 3-{3-[2-(benzyloxy)-5-chloro phenyl]-pyridin-4-yl}benzoic acid ethyl ester but using 2-(benzyloxy)phenylboronic acid instead of 2-(benzyloxy)-5-chlorophenylboronic acid. LC/MS t=3.62, [MH+] 410.1

### 4-[2-(Benzyloxy)-5-chloro-phenyl]-3-bromopyridine

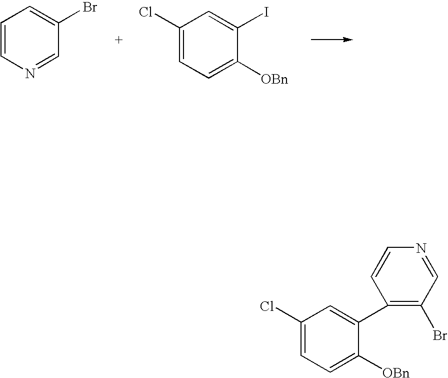

The title compound was prepared using an analogous procedure to that used for the preparation of ethyl 3-(3-bromopyridin-4-yl)benzoate but using 2-(benzyloxy)-5-chloro-iodobenzene instead of ethyl 3-iodobenzoate. LC/MS t=3.71, [MH+] 375.9, 377.8.

### 3-{4-[2-(Benzyloxy)-5-chloro-phenyl]-pyridin-3-yl}benzoic Acid Ethyl Ester

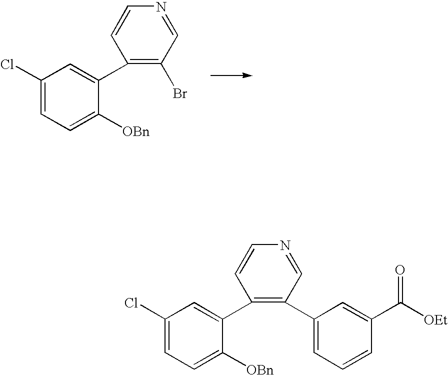

The title compound was prepared using an analogous procedure to that used for the preparation of 3-{3-[2-(benzyloxy)-5-chloro-phenyl]-pyridin-yl}-benzoic acid ethyl ester but using 4-[2-(benzyloxy)-5-chlorophenyl]-3-bromopyridine instead of ethyl 3-(3-bromopyridin-4-yl)benzoate and 3-(ethoxycarbonyl)-phenylboronic acid instead of 2-(benzyloxy-chloro-phenylboronic acid. LC/MS t=3.80, [MH+] 444.0, 446.0.

### 3-[2-(Benzyloxy)-5 chloro-phenyl]-2-bromopyridine

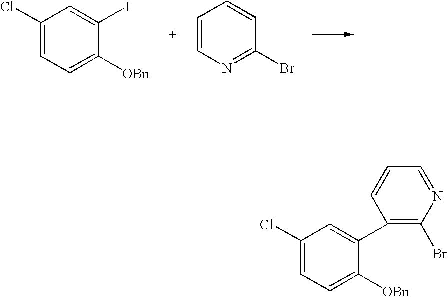

Was prepared sing an analogous procedure to that used for the preparation of 3-(3-bromopyridin-4-yl)-benzoic acid ethyl ester but using 2-bromopyridine instead of 3-bromopyridine and 2-(benzyloxy)-5-chloro-iodobenzene instead of ethyl 3-iodobenzoate. LC/MS t=3.69, [MH+] 375.9, 377.9.

### 3-{3-[2-(Benzyloxy)-5-chloro-phenyl]-pyridin-2-yl}-benzoic Acid Ethyl Ester

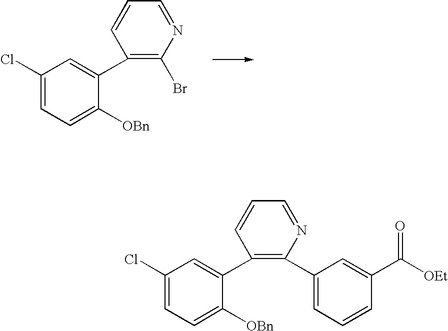

The title compound was prepared using an analogous procedure to that used for the preparation of 3-{3-[2-(benzyloxy)-5-chloro-phenyl]-pyridin-4-yl}-benzoic acid ethyl ester but using 3-[2-(benzyloxy)-5-chloro-phenyl]-2-bromopyridine instead of ethyl 3-(3-bromopyridin-4-yl)benzoate and 3-ethoxycarbonyl)phenylboronic acid instead of 2-(benzyloxy)-5-chlorophenylboronic acid. LC/MS t=3.85, [MH+] 444.1.

### Example 9

### 3-{3-[2-(Benzyloxy)-5-chloro-phenyl]-pyridin-4-yl}-benzoic Acid

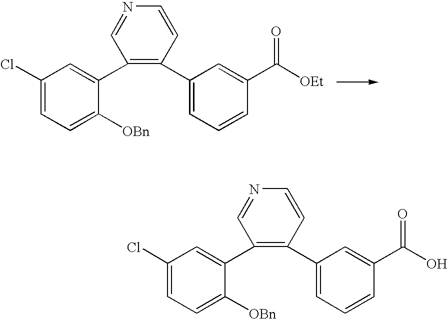

A solution of 3-{3-[2-(benzyloxy)-5-chloro-phenyl]-pyridin-4-yl}-benzoic acid ethyl ester (175 mg, 0.39 mmol) in ethanol (6 ml) and 2M sodium hydroxide (2 ml) was left at room temperature for 3 hours. The solvent was evaporated and the residue dissolved in water and washed with ether. The aqueous phase was acidified with acetic acid and extracted with ethyl acetate which was dried (magnesium sulphate) evaporated and the residue triturated with ether to give the title compound (136 mg) as an off-white solid.

LC/MS t=3.44, [MH+] 416.0, 418.0.

The following compounds were prepared by an analogous hydrolysis procedure from an appropriate ester intermediate.

| EXAMPLES | COMPOUND NAME | LC/MS |
|---|---|---|
| Example 10 | 3-{3-[2-(Benzyloxy)- | t = 3.22 |
| | phenyl]-pyridin-4-yl}- | [MH+] 382.1 |
| | benzoic acid | |
| Example 11 | 3-{4-[2-(Benzyloxy)-5- | t = 3.49 |
| | chloro-phenyl]-pyridin- | [MH+] 416.0, |
| | 3-yl}-benzoic acid | 418.0 |
| Example 12 | 3-{3-[2-(Benzyloxy)-5- | t = 3.57 |
| | chloro-phenyl]-pyridin- | [MH+] 416.1, |
| | 2-yl}-benzoic acid | 418.1 |

General procedure A

### A (i) 4-(Benzyloxy)benzotrifluoride

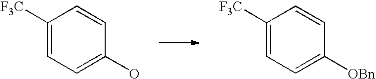

A solution of 4-hydroxybenzotrifluoride (8.55 g, 52.78 mmol) in acetone (200 ml) was treated with benzyl bromide (9.87 g, 6.86 ml, 58.05 mmol) and potassium carbonate (10.94 g, 79.16 mmol). The mixture was stirred and heated to reflux under nitrogen for 3 hours. After cooling, diethyl ether (400 ml) and water (400 ml) were added. The layers were separated and the aqueous phase re-extracted with diethyl ether (100 ml). The combined organic layers were washed with water, dried (MgSO₄) and the solvent removed in vacuo to leave the title compound (12.719 g 95%) as a white solid.

¹H NMR (CDCl₃) δH: 5.11 (2H, s), 7.03 (2H, d, J=9 Hz), 7.34-7.44 (5H, m), 7.55 (2H, d, J=9 Hz).

### A (ii) 4-(Benzyloxy)-3-iodobenzotrifluoride

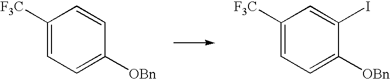

A solution of 4-(benzyloxy)benzotrifluoride (12.71 g, 50.4 mmol) in acetonitrile (300 ml) was stirred under nitrogen and 1-(chloromethyl)-4-fluoro-1,4-diazoniabicyclo[2.2.2]octane bis(tetrafluoroborate) (17.75 g, 50.4 mmol) and iodine (6.4 g, 25.2 mmol) added. The mixture was stirred at room temperature for 88 h. The solvent was evaporated and the residue partitioned between ethyl acetate (400 ml) and water (400 ml). The organic layer was was washed with water, dried (MgSO₄) and evaporated to an orange oil which was purified by flash chromatography on silica gel with 5% ethyl acetate in isohexane as eluant to give the title compound as an orange oil (15.07 g, 79%).

¹H NMR (CDCl₃) δH: 5.21 (2H, s), 6.89 (1H, d J=9 Hz), 7.32-7.55 (6H, m), 8.04 (1H, d, J=2 Hz).

### A (iii): 2-(Benzyloxy-5-(trifluoromethyl)-benzeneboronic Acid

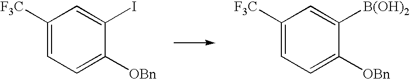

A solution of 4-(benzyloxy-3-iodobenzotrifluoride (15.07 g, 39.85 mmol) in tetrahydrofuran (200 ml) was cooled to −40° C. with stirring under nitrogen. 2M isopropylmagnesium chloride in diethyl ether (39.85 ml, 79.7 mmol) was added dropwise and the mixture stirred at −40° C. for 40 minutes, then cooled to −75° C. Trimethyl borate (8.3 g, 9.2 ml, 79.7 mmol) was added at −75° C. over 10 minutes and the reaction stirred and allowed to reach 0° C. over 1 h. 1M hydrochloric acid (200 ml) was added and the mixture stirred vigorously for 1 h. The layers were separated and the aqueous layer extracted with diethyl ether (100 ml). The combined organic layers were washed with water, dried (MgSO₄) and evaporated. The residue was flash chromatographed on silica gel with 5-20% ethyl acetate in isohexane as eluant to give the title compound (7.71 g, 65%) as a white solid.

¹H NMR (CDCl₃) δH: 5.20 (2H, s), 5.79 (2H, s), 7.05 (1H, d, J=9 Hz), 7.39-7.44 (5H, m), 7.68 (1H, dd J=2 Hz, J=9 Hz), 8.15 (1H, d, J=2 Hz).

### 4-(4-Fluorobenzyloxy)-benzotrifluoride

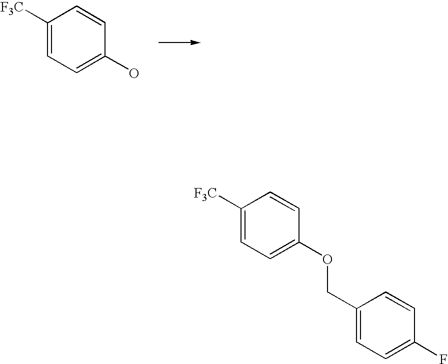

Prepared by general procedure A(i) but using 4-fluorobenzyl bromide instead of benzyl bromide.

¹H NMR (CDCl₃) δH, 5.07 (2H, s), 7.02 (2H, d, J=9 Hz), 7.07-7.11 (2H, m), 7.39-7.42 (2H, m), 7.52 (2H, d, J=9 Hz).

### 2-(4-Fluorobenzyloxy)-5-(trifluoromethyl)-iodobenzene

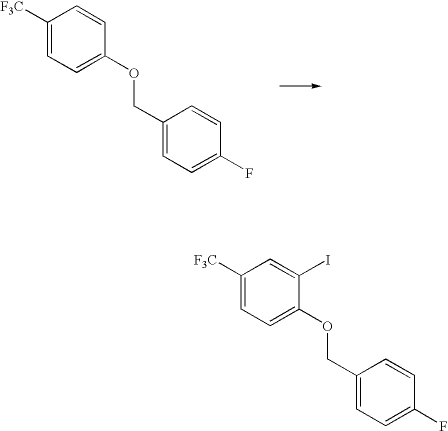

Prepared by general procedure A(ii) but using 4-(4-fluorobenzyloxy)-benzotrifluoride instead of 4-(benzyloxy)benzotrifluoride.

¹H NMR (CDCl₃) δH, 5.16 (2H, s), 6.88 (1H, d, J=9 Hz), 7.08-7.13 (2H, m), 7.44-7.48 (2H, m), 7.54-7.57 (1H, dd, J=2 Hz, J=9 Hz), 8.04 (1H, d, J=2 Hz).

### 2-(4-Fluorobenzyloxy)-5-(trifluoromethyl)-benzeneboronic Acid

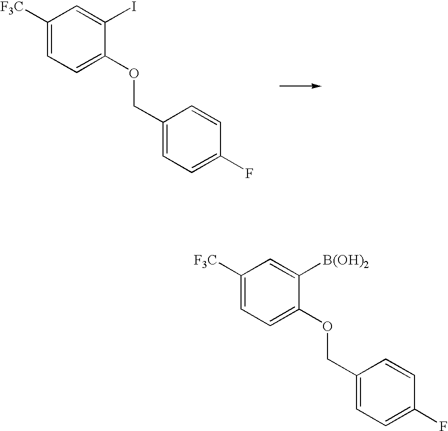

Prepared by general procedure A(iii) but using 4-(4-fluorobenzyloxy)-3-iodobenzotrifluoride instead of 4-(benzyloxy)-3-iodobenzotrifluoride.

¹H NMR (DMSO d₆) δH, 5.22 (2H, s), 7.20-7.26 (3H, m), 7.54-7.58 (2H, m), 7.71 (1H, d, J=9 Hz), 7.75 (1H, s), 8.03 (2H, s).

### 4-(2,4-Difluorobenzyloxy)-benzotrifluoride

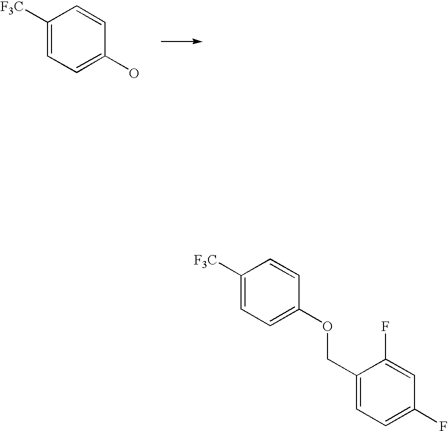

Prepared by general procedure A(i) but using 2,4-difluorobenzyl bromide instead of benzyl bromide.

¹H NMR (CDCl₃) δH: 5.12 (2H, s), 6.89 (2H, dt, J=2 Hz, J=9 Hz), 7.02-7.05 (2H, d, J=9 Hz), 7.33-7.49 (1H, q, J=8 Hz, J=15 Hz), 7.56 (2H, d, J=9 Hz)

### 4-(2,4-Difluorobenzyloxy)-5-iodobenzotrifluoride

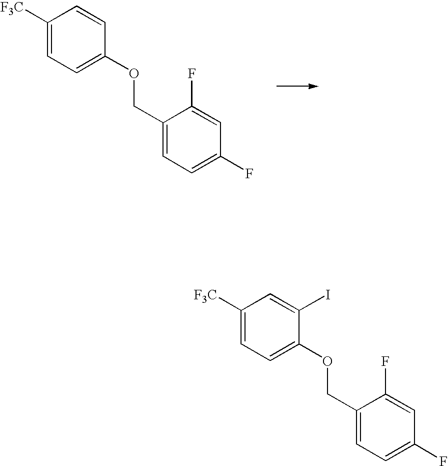

Prepared by general procedure A(ii) but using 4-(2,4-difluorobenzyloxy)benzotrifluoride instead of 4-(benzyloxy)benzotrifluoride.

¹H NMR (CDCl₃) δH: 5.21 (2H, s), 6.84-6.95 (3H, m), 7.55-7.65 (2H, m), 8.04 (1H, s)

### 2-{2,4-Difluorobenzyloxy)-(trifluoromethyl)-benzeneboronic Acid

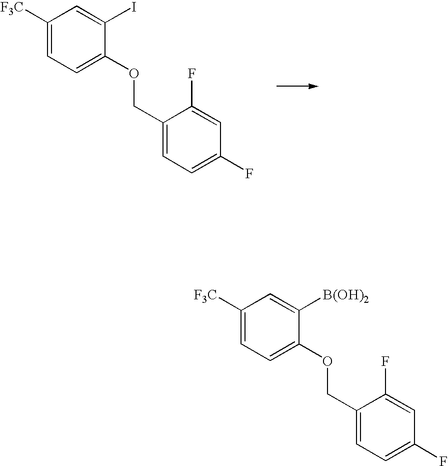

Prepared by general procedure A(iii) but using 4-(2,4-difluorobenzyloxy)-3-iodobenzotrifluoride instead of 4-(benzyloxy)-3-iodobenzotrifluoride.

¹H NMR (DMSO d₆) δH, 5.26 (2H, s), 7.16 (1H, dt J=2 Hz, J=9 Hz) 7.27 (1H, d, J=9 Hz), 7.33 (1H, dt, J=2 Hz, J=9 Hz), 7.68-7.75 (3H, m), 8.01 (2H, s).

### 3-iodo-5-nitrobenzoic Acid

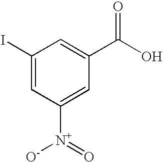

A solution of sodium nitrite (1.66 g, 24 mmol) was added over 10 min. to a stirred suspension of 3-amino-5-nitrobenzoic acid (3.64 g, 20 mmol) in 10 ml of conc. HCl at 0° C. Stirred for 1 h then added in portions over 2 min. to a stirred solution of potassium iodide (8.3 g, 50 mmol) in 30 ml of water at 0° C. The mixture was then allowed to warm to room temperature over 2 h and extracted with ethyl acetate. The organic layer was washed with sodium thiosulphate solution, dried and evaporated. The residue was purified by chromatography on a silica gel with 10% methanol in ethyl acetate, as eluant, to give the title compound as a red solid.

¹H NMR (DMSO d₆) δH: 8.55 (1H, s), 8.57 (1H, s), 8.71 (1H, s).

### Ethyl 3-iodo-5-nitrobenzoate

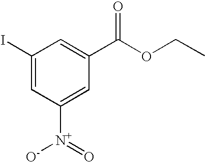

3-iodo-5-nitrobenzoic acid (9.3 g, 0.0317 mol) and thionyl chloride (20 ml) were heated to reflux for 3 h. After cooling, the solvent was removed in vacuo and the residue was then redissolved in toluene and evaporated to dryness. Toluene (30 ml) and ethanol (120 ml) were added dropwise over a period of 30 min. The mixture was stirred at room temperature overnight. The solvent was then dried (MgSO₄) and evaporated and the residue was purified by chromatography on silica gel with 5% ethyl acetate in iso-hexane as eluant, to give the title compound as a yellow solid (8 g, 78%).

¹H NMR (CDCl₃) δH, 1.44 (3H, t, J=7.1 Hz), 4.45 (2H, q, J=7.1 Hz), 8.67 (1H, s), 8.72 (1H, s), 8.80 (1H, s).

### Ethyl 3-amino-5-iodobenzoate

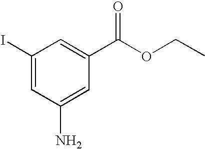

Zinc powder (13.6 g, 0.21 mol) was added over 20 min. to a solution of ethyl 3-iodo-5-nitrobenzoate (7.4 g, 0.023 mol) in acetic acid (200 ml) at 0° C. The reaction mixture was stirred for 1 hour then filtered and evaporated. The residue was dissolved in diethyl ether and 1M NaOH solution. The layers were separated and the organic layer was then extracted with 1M HCl solution (3×20 ml). The combined acidic layers were basified with 1M NaOH solution and extracted with diethyl ether (2×30 ml). The combined organic layers were dried (MgSO₄) and concentrated. Purification was carried out on silica gel with 15% ethyl acetate in iso-hexane as eluant to give the title compound.

¹H NMR (CDCl₃) δH: 1.36 (3H, t, J=7.1 Hz), 3.94 (2H, bs), 4.33 (2H, q, J=7.1 Hz), 7.17 (1H, s), 7.28 (1H, s), 7.69 (1H, s).

### Ethyl 3-(acetylamino)-5-iodobenzoate

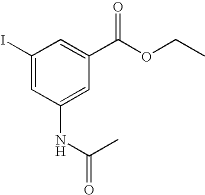

Acetic anhydride (0.92 ml, 0.0097 mol) was added to a solution of ethyl 3-amino-5-iodobenzoate (2.57 g, 0.0089 mol) and triethylamine (1.35 ml, 0.0097 mol) in CH₂Cl₂ and stirred at room temperature for 4 h. More acetic anhydride (0.92 ml) and triethylamine (1.35 ml) were added and the mixture stirred for other 2 h. The reaction mixture was diluted with more CH₂Cl₂ and washed sequentially with 1M HCl and 1M NaOH solution, dried and evaporated. The residue was triturated with dichloromethane and iso-hexane to yield the title compound (1.2 g, 41%) as a white solid.

¹H NMR (CDCl₃) δH: 1.38 (3H, t, J=7.1 Hz), 2.19 (3H, s), 4.36 (2H, q, J=7.1 Hz), 7.91 (1H, s), 8.1 (1H, s), 8.34 (1H, s).

### Ethyl 3-(acetylamino)-5-(3-bromo pyridinyl)benzoate

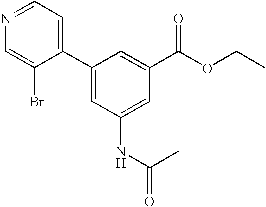

1.6M BuLi in hexanes (3 ml, 0.0048 mol) was added over 10 min. to a solution of diisopropylamine (0.68 ml, 0.0048 mol) in THF (10 ml) at −78° C. under nitrogen. The solution was stirred for 10 min. then allowed to warm to room temperature and added to a solution of 3-bromopyridine (0.39 ml, 0.004 mol) in THF (6 ml) at −95° C. over 15 min. The reaction mixture was stirred for 25 min before a solution of 0.5M ZnCl₂ (8.1 ml) in THF was slowly added. The solution was then warmed to room temperature and ethyl 3-(acetylamino)-5-iodobenzoate (0.5 g, 0.0015 mol) and Pd(PPh₃)₄ in THF (10 ml) were added. The mixture was refluxed for 3 hours, cooled and evaporated. The residue was dissolved in diethyl ether and washed with 1M NaOH solution. The organic layer was dried (MgSO₄), filtered and concentrated to give an orange solid that was crystallized from iso-hexane and dichloromethane to yield the title compound as a white solid.

LC/MS: Rt=2.95 [MH+]=365, 366 [MH−]=362, 364.

### General Procedure B

### 3-{3-[2-Benzyloxy)-5-(trifluoromethyl)-phenyl]-pyridin-4-yl}-5-(acetylamino)-benzoic Acid Ethyl Ester

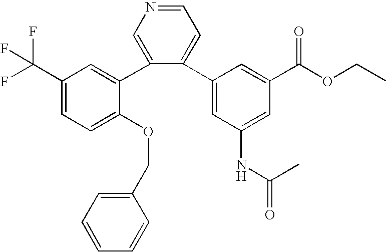

2-(Benzyloxy)-5-(trifluoromethyl)benzeneboronic acid (47 mg, 0.158 mmol), Pd(PPh₃)₄ (19 mg, 0.015 mmol), potassium carbonate (177 mg, 1.27 mmol) and ethyl 3-(acetylamino)-5-(3-bromo-pyridin 4-yl)benzoate (70 mg, 0.19 mmol) in toluene-ethanol (1:16 ml) were stirred at 90° C., under nitrogen overnight. Upon cooling, the reaction mixture was poured into water and extracted with ethyl acetate (3×10 ml). The combined organic layers were dried (MgSO₄), and concentrated. The residue was purified on an SPE column using iso-hexane containing a gradient of ethyl acetate (50-90%) to give the title compound as a white solid. LC/MS: Rt=3.52 [MH+]=535, 536 [MH−]=533, 534.

### 3-{3-[2-(4-Fluoro-benzyloxy)-5-(trifluoromethyl)-phenyl]-pyridin-4-yl}-5-(acetylamino) benzoic Acid Ethyl Ester

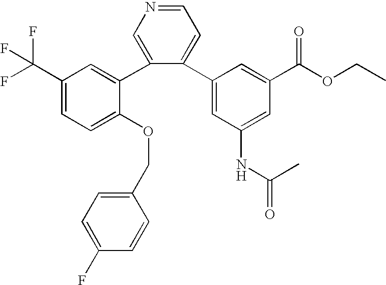

Prepared according to general procedure B but using 2-(4-fluorobenzyloxy 5-(trifluoromethyl)-benzeneboronic acid instead of 2-(benzyloxy)-5-(trifluoromethyl)-benzeneboronic acid. LC/MS: Rt=3.57 [MH+]=571, 572 [MH−/]=569, 570.

### 3-{3-[2-(2,4-Difluoro-benzyloxy)-5-(trifluoromethyl)phenyl]-pyridin-4-yl}-5-(acetylamino) benzoic Acid Ethyl Ester

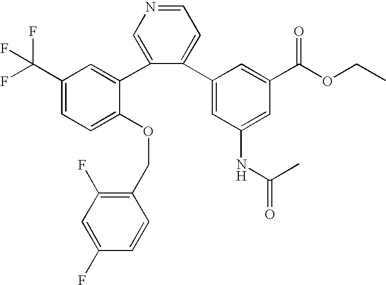

Prepared according to general procedure L but using 2-(2,4-Difluorobenzyloxy-5-(trifluoromethylbenzeneboronic acid instead of 2-benzyloxy-5-(trifluoromethyl)benzeneboronic acid. LC/MS: Rt=3.55 [MH+]=553, 554 [MH−]=551, 552.

### 3-{3-[2-(Benzyloxy)-phenyl]-pyridin-4-yl}-5-(acetylamino)benzoic Acid Ethyl Ester

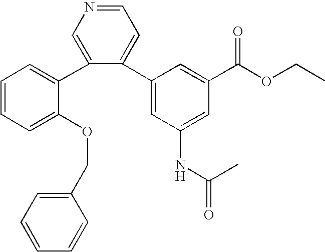

Prepared according to general procedure B but using 2-(benzyloxy)phenylboronic acid instead of 2-(benzyloxy-5-(trifluoromethyl)benzeneboronic acid. LC/MS: Rt=3.28 [MH+]=467, 468 [MH−]=465, 466

### General Procedure C

### Example 13

### 3-{3-[2-(Benzyloxy)-54(trifluoromethyl)-phenyl]-pyridin-4-yl}-5-(acetylamino)-benzoic Acid

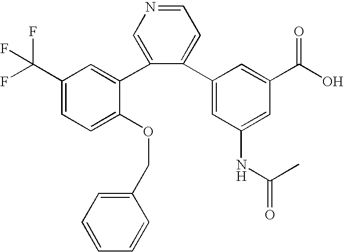

3-{3-[2-(Benzyloxy)-5-(trifluoromethyl)-phenyl]-pyridin-4-yl}-5-(acetylamino)benzoic acid ethyl ester (56 mg, 0.105 mmol) and NaOH (16 mg, 0.42) were dissolved in ethanol (3 ml) and H₂O (1 ml). The mixture was heated at 60° C. for 3 hours. The solution was then diluted with water and acidified with acetic acid. The mixture was extracted with ethyl acetate and the organic solution was dried over magnesium sulphate, filtered and evaporated to give the title compound.

LC/MS: Rt=3.21 [MH+]=507, 508 [MH−]=505, 506

### Example 14

### 3-{3-[2-(4-Fluoro-benzyloxy)-5-(trifluoromethyl)-phenyl]-pyridin-4-yl}-5-(acetylamino)-benzoic Acid

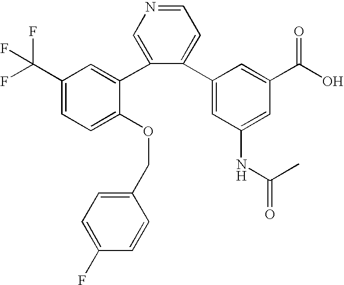

Prepared according to general procedure C except 3-{3-[2-(benzyloxy)-5-trifluoromethyl)-phenyl]-pyridin-4-yl}5-(acetylamino)-benzoic acid ethyl ester was replaced with 3-{3-[2-(4-fluoro-benzyloxy)-5-(trifluoromethyl)-phenyl]-pyridin-4-yl}-5-(acetylamino)benzoic acid ethyl ester.

LC/MS: Rt=3.25 [MH+]=525, 526 [MH−]=523, 524.

### Example 15

### 3-{3-[2-(2,4-Difluoro-benzyloxy)-5-(trifluoromethyl)-phenyl]-pyridin-4-yl}-5-(acetylamino)-benzoic Acid

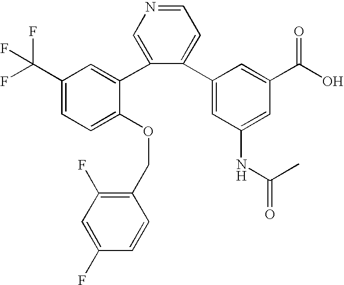

Prepared according to general procedure C except 3-{3-[2-(benzyloxy-5-(trifluoromethyl)phenyl]pyridin-4-yl}5-(acetylamino)-benzoic acid ethyl ester was replaced with 3-{3-[2-(2,4-difluoro-benzyloxy)-5-(trifluoromethyl)-phenyl]-pyridin-4-yl}-5-(acetylamino)-benzoic acid ethyl ester.

LC/MS: Rt=3.27 [MH+]=543, 544 [MH−]=541, 542.

### Example 16

### 3-{3-[2-(Benzyloxy)-phenyl]-pyridin 4-yl}-5-(acetylamino)-benzoic Acid

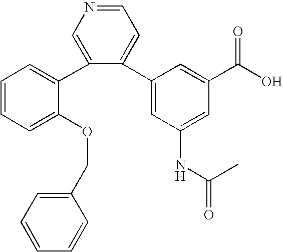

Prepared according to general procedure C except 3-{3-[2-(benzyloxy)-5-(trifluoromethyl)phenyl]-pyridin-4-yl}-5-(acetylamino)-benzoic acid ethyl ester was replaced with 3-{3-[2-(Benzyloxy)phenyl-pyridin-4-yl}-5-(acetylamino)-benzoic acid ethyl ester.

LC/MS: Rt=2.88 [MH+]=439, 440 [MH−]=437, 438.

### Example 17

### 6-{1-[2-(Benzyloxy)-5-chloro-phenyl]-5-methyl-1H-pyrrol-2-yl}-2-pyridinecarboxylic Acid, Sodium Salt

### a) 1-(6-bromo-pyridin-2-yl)-pentane-1,4-dione

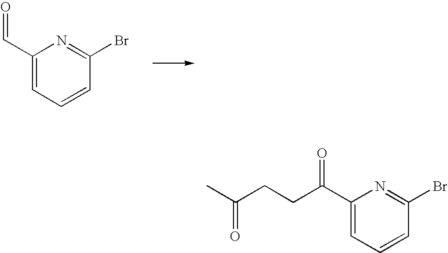

6-Bromo-2-pyridine carboxaldehyde (1.0231 g, 5.50 mmol), methyl vinyl ketone (0.55 mL, 6.59 mmol) and 3-ethyl-5-(2-hydroxyethyl)-4-methylthiazolium bromide (0.329 g, 1.31 mmol) were heated in a mixture of ethanol (1.8 mL) and triethylamine (0.68 mL) at 90° C. for 2.25 hours. Upon cooling, the mixture diluted with saturated ammonium chloride and extracted with ethyl acetate. The combined extracts were washed with saturated sodium bicarbonate, dried (Na₂SO₄), filtered and concentrated. The residue was purified by chromatography on silica gel with iso-hexane containing ethyl acetate (10-15%), as eluant, to give the title compound (1.196 g, 85%). LC/MS Rt=2.36 min.

### b) 2-[1-(2-Benzyloxy-5-chloro-phenyl)-5-methyl-1H-pyrrol-2-yl]-6-bromo-pyridine

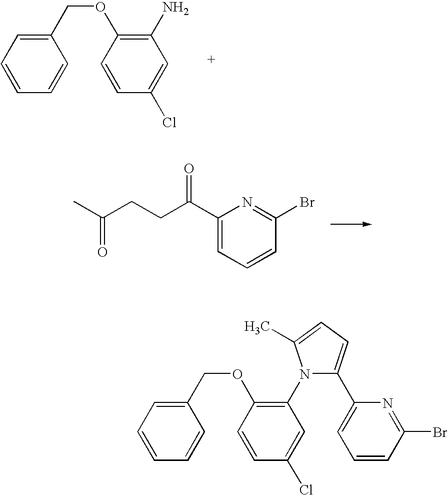

2-Benzyloxy-5-chloro-aniline (0.374 g, 1.6 mmol), 1-(6-bromo-pyridin-2-yl)-pentane-1,4-dione (0.406 g, 1.6 mmol) and p-TSA (0.036 g) were heated in NMP at 150° C. in a microwave reactor for 45 minutes. The mixture was diluted with saturated ammonium chloride and extracted twice with diethyl ether. The combined extracts were washed with saturated sodium bicarbonate, dried (Na₂SO₄), filtered and concentrated. The residue was purified by chromatography on silica gel with iso-hexane containing ethyl acetate (2-3%) as elueant, to give the title compound (0.221 g, 31%).

LC/MS Rt=4.24 min, [MH⁺] 455, 457.

### c) 6-{1-[2-(Benzyloxy)-5-chloro-phenyl]-5-methyl-1H-pyrrol-2-yl}-2-pyridinecarboxylic Acid Ethyl Ester

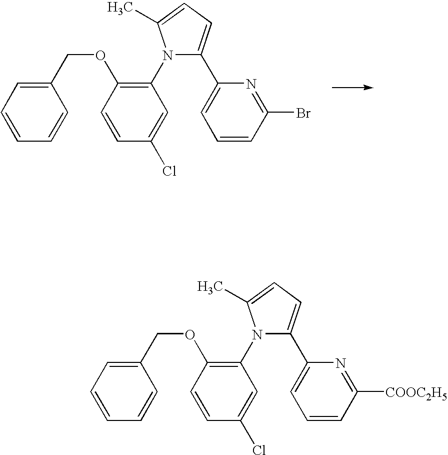

Carbon monoxide gas was introduced into a mixture of 2-[1-(2-benzyloxy-5-chloro-phenyl)-5-methyl-1H-pyrrol-2-yl]bromo-pyridine (219 mg), triethylamine (0.7 ml), dichlorobis(triphenylphosphine)palladium(II) (18 mg) and ethanol (2.5 ml). The mixture was stirred under reflux for 18 hours and more palladium catalyst (50 mg) and triethylamine (0.7 ml) added. More carbon monoxide gas was introduced and the mixture stirred under reflux for 72 hours, during which time carbon monoxide was introduced twice more. The mixture was concentrated under reduced pressure and the residue partitioned between water (5 ml) and ethyl acetate (5 ml). The aqueous layer was extracted with ethyl acetate (2×5 ml), the combined extracts washed with brine (5 ml), dried (MgSO₄), evaporated and the residue purified by Biotage chromatography eluting with 10% ethyl acetate in isohexane to afford the title compound (42 mg). LC/MS Rt=4.1 min, [MH⁺] 447, 449.

### d) 6-{1-[2-(Benzyloxy)₅-chloro-phenyl]-5-methyl-1H-pyrrol-2-yl}-2-pyridinecarboxylic Acid, Sodium Salt

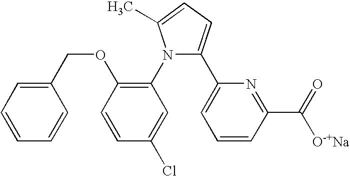

A solution of 6-{1-[2-(benzyloxy)-5-chloro-phenyl]-5-methyl-1H-pyrrol-2-yl}-2-pyridinecarboxylic acid ethyl ester (41.5 mg) in 2N sodium hydroxide solution (0.2 ml) and ethanol (1 ml) was stirred under reflux for 2 hours. The solution was concentrated under reduced pressure and the residue dissolved in water (2 ml). The solution was washed with ether (2 ml) and the ether layer was then extracted with water (2×1 ml). The combined aqueous layers were concentrated under reduced pressure to ca. 2 ml and the solution extracted with ethyl acetate (3×2 ml). The combined organic extracts were dried (MgSO₄), evaporated and the residue washed with isohexane-ether and dried in vacuo at 50° C. to afford the title compound (28.5 mg). LC/MS Rt=3.7 min, [MH⁺]419, 421.

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]_{I}) in response to activation of EP₁, or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]_{I} produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁, or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (plC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]—PGE₂) for binding to the human EP₁ receptor.

This assay utilises Chinese hamster ovary-K1 (CHO—K1) cells into which a stable vector containing the EP₁, cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at 80° C. until required.

For the binding assay the cell membranes, competing compounds and [3H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques (GraphPad Prism 3) to determine the concentration of compound producing 50% inhibition of specific binding (IC₅₀).

By application of these techniques, compounds of the examples had an antagonist plC₅₀ value of >6.0 at EP₁ receptors and plC₅₀ value of <6.0 at EP₃ receptors.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 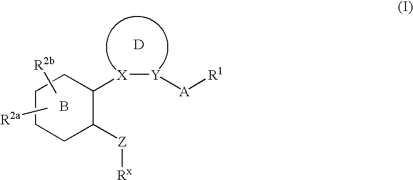
wherein:
A represents an optionally substituted aryl, or an optionally substituted 5- or 6-membered heterocyclyl ring, or an optionally substituted bicyclic heterocyclyl group;
B represents a phenyl or pyridyl ring;
D represents an optionally substituted 5- or 6-membered heterocyclyl ring containing one or two heteroatoms selected from N, S and O, wherein X and Y are each independently selected from N and C;
Z represents O, S, SO, or SO₂;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷,
R⁷ represents hydrogen, optionally substituted alkyl, optionally substituted heteroaryl or optionally substituted aryl;
Q^{a} and Q^{b} are each independently selected from hydrogen and CH₃;
wherein when A is a 6-membered ring the R¹ substituent and the D ring are attached to carbon atoms 1,2-, 1,3- or 1,4-relative to each other, and when A is a five-membered ring or bicyclic heterocyclyl group the R¹ substituent and the D ring are attached to substitutable carbon atoms 1,2- or 1,3-relative to each other;
and derivatives thereof;
provided that D is not imidazolyl, thienyl, 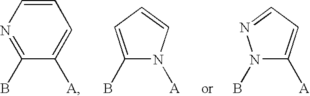
wherein A and B are as hereinbefore defined.

2. A compound according to claim 1 wherein D is selected from 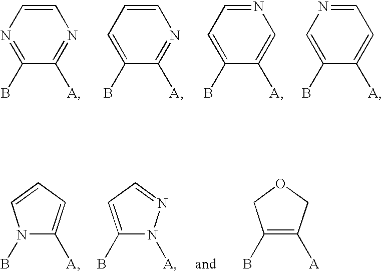
all of which may be optionally substituted.

3. A compound according to claim 1 wherein A in is selected from pyridyl or optionally substituted phenyl.

4. A compound according to claim 1 wherein R¹ is CO₂H.

5. A compound selected from:
3-{1-[2-(benzyloxy)-phenyl]-5-methyl-1H-pyrrol-2-yl}-benzoic acid;
3-{1-[2-(benzyloxy)-5-chloro-phenyl]-5-methyl-1H-pyrrol-2-yl}-benzoic acid;
3-{1-[2-(benzyloxy)-5-bromo-phenyl]-5-methyl-1H-pyrrol-2-yl}-benzoic acid;
3-{5-[2-(benzyloxy)-phenyl]-1H-pyrazol-1-yl}-benzoic acid;
3-{5-[2-(benzyloxy)-5-chloro-phenyl]-1H-pyrazol-1-yl}-benzoic acid;
3-{3-[2-(benzyloxy)-5-chloro-phenyl]-pyrazin-2-yl}-benzoic acid;
3-{4-[2-(benzyloxy)-5-chloro-phenyl]-2-oxo-2,5-dihydro-furan-3-yl}-benzoic acid;
3-{3-[2-(benzyloxy)-5-chloro-phenyl]-2-oxo-Z 5-dihydro-furan-4-yl}-benzoic acid;
3-{3-[2-(benzyloxy)-5-chloro-phenyl]-pyridin-4-yl}-benzoic acid;
3-{3-[2-(benzyloxy)-phenyl]-pyridin-4-yl}-benzoic acid;
3-{4-[2-(benzyloxy)-5-chloro-phenyl]-pyridin-3-yl}-benzoic acid;
3-{3-[2-(benzyloxy)-5-chloro-phenyl]-pyridin-2-yl}-benzoic acid;
3-{3-[2-(benzyloxy)-5-(trifluoromethyl)-phenyl]-pyridin-4-yl}-5-(acetylamino)-benzoic acid;
3-{3-[2-(4-fluoro-benzyloxy)-5-(trifluoromethyl)-phenyl]-pyridin-4-yl}-5-(acetylamino)-benzoic acid;
3-{3-[2-(2,4-difluoro-benzyloxy)-5-(trifluoromethyl)-phenyl]-pyridin-4-yl}-5-(acetylamino)-benzoic acid;
3-{3-[2-(benzyloxy)-phenyl]-pyridin-4-yl}-5-(acetylamino)-benzoic acid; and
6-{1-[2-(benzyloxy)-5-chloro-phenyl]-5-methyl-1H-pyrrol-2-yl}-2-pyridinecarboxylic acid;
and derivatives thereof.

6. A pharmaceutical composition comprising a compound according to claim 1 or a pharmaceutically acceptable derivative thereof together with a pharmaceutical carrier and/or excipient.

7.-8. (canceled)

9. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

10. A method of treating a human or animal subject suffering from a pain, inflammatory, immunological, bone, neurodegenerative or renal disorder, which method comprises administering to said subject an effective amount of a compound according to claim 1 or a pharmaceutically acceptable derivative thereof.

11. A method of treating a human or animal subject suffering from inflammatory pain, neuropathic pain or visceral pain which method comprises administering to said subject an effective amount of a compound according to claim 1 or a pharmaceutically acceptable derivative thereof.

12.-14. (canceled)

15. The method of claim 9 wherein the subject is human.

16. The method of claim 10 wherein the subject is human.

17. The method of claim 11 wherein the subject is human.

18. A method of mediating EP₁ receptors, comprising the step of administering an effective amount of a compound according to claim 1 or a pharmaceutically acceptable derivative thereof.
